SEQUENCE LISTING (10 sequences)

SEQ ID NO: 1 - DNA, 20 bp, unidentified
  source 1..20: mol_type = other DNA
ggaagggctcatgaccacag

SEQ ID NO: 2 - DNA, 21 bp, unidentified
  source 1..21: mol_type = other DNA
acagtcttctgggtggcagtg

SEQ ID NO: 3 - DNA, 23 bp, unidentified
  source 1..23: mol_type = other DNA
aagggacacagaggtttcagtgg

SEQ ID NO: 4 - DNA, 24 bp, unidentified
  source 1..24: mol_type = other DNA
cagcaccagtagcaccatcatttc

SEQ ID NO: 5 - DNA, 21 bp, unidentified
  source 1..21: mol_type = other DNA
gtgaagaagaggacagcactg

SEQ ID NO: 6 - DNA, 18 bp, unidentified
  source 1..18: mol_type = other DNA
cccattcccaccatcacc

SEQ ID NO: 7 - DNA, 17 bp, unidentified
  source 1..17: mol_type = other DNA
gtccagcacgaggctca

SEQ ID NO: 8 - DNA, 17 bp, unidentified
  source 1..17: mol_type = other DNA
tcgccttcgtggtcctc

SEQ ID NO: 9 - DNA, 20 bp, unidentified
  source 1..20: mol_type = other DNA
tgtcagtcaaagcaagcccg

SEQ ID NO: 10 - DNA, 24 bp, unidentified
  source 1..24: mol_type = other DNA
ttaggacgctcataagtgtcaccc

(19) United States
(12) Patent Application Publication
(10) Pub. No.: US 2025/0170143 A1
(43) Pub. Date: May 29, 2025

(54) METHODS OF TREATING OCULAR FIBROTIC PATHOLOGIES

(71) Applicant: Mayo Foundation for Medical Education and Research, Rochester, MN (US)

(72) Inventors: Andrew J. Haak, Rochester, MN (US); Sophie Bakri, Rochester, MN (US)

(21) Appl. No.: 18/844,205

(22) PCT Filed: Apr. 13, 2023
(86) PCT No.: PCT/US2023/065746

§ 371 Date: Sep. 5, 2024

Related U.S. Application Data
(60) Provisional application No. 63/331,154, filed on Apr. 14, 2022.

(51) Int. Cl. A61K 31/553; A61K 45/06; A61P 27/02; C07D 498/04; C07D 498/14

(52) U.S. Cl. CPC A61K 31/553; A61K 45/06; A61P 27/02; C07D 498/04; C07D 498/14

(57) ABSTRACT

The present application provides compounds and methods for treating ocular fibrotic pathologies, including using dopamine D2 receptor antagonists for treating proliferative vitreoretinopathy.

### CLAIM OF PRIORITY

This application claims priority to U.S. Patent Application Ser. No. 63/331,154, filed on Apr. 14, 2022.

### TECHNICAL FIELD

This invention relates to compounds and methods useful in treating ocular fibrotic pathologies, such as proliferative vitreoretinopathy, diabetic retinopathy, and age-related macular degeneration and many others.

### BACKGROUND

Fibrosis affects many parts of the eye and is a pathway in many eye diseases, and also can be an unwanted complication of treatment. Fibrosis occurs in diabetic retinopathy, epiretinal membranes, proliferative vitreoretinopathy, macular degeneration, choroidal neovascularization and any eye diseases that result from angiogenesis, trauma, inflammation, or infection. After surgery for glaucoma (e.g., trabeculectomy and filtering procedures), fibrosis can result in surgical failure and need for an anti-fibrotic intervention.

### SUMMARY

Retinal pigmented epithelial (RPE) cells play an important role in retinal fibrotic diseases such as proliferative vitreoretinopathy (PVR). Experimental results of this disclosure show that dopamine receptor DRD2 is one of the dominant dopaminergic receptors expressed in RPE cells. RPE cells produce dopamine and exhibit elevated dopamine production in the presence of fibrosis-inducing cytokine TGFβ. Experimental results presented in this disclosure advantageously demonstrate that treatment of RPE cells with D2 dopamine receptor antagonists inhibits profibrotic gene expression, migration, proliferation, and fibronectin deposition and is thus effective in treating retinal fibrotic pathologies including PVR.

In one general aspect, the present disclosure provides a method of treating or preventing an ocular fibrotic pathology, the method comprising administering to a subject in need thereof a therapeutically effective amount of a dopamine receptor D2 (DRD2) antagonist, or a pharmaceutically acceptable salt thereof.

In some embodiments, the ocular fibrotic pathology is selected from: proliferative vitreoretinopathy (PVR), diabetic retinopathy, ischemic retinopathy, age-related macular degeneration (ARMD), dry ARMD, neovascular ARMD, retinal neovascularization, choroidal neovascularization, epiretinal membrane, keratitis, pterygia, pingueculae, retinopathy of prematurity, glaucoma (including neovascular glaucoma, open-angle glaucoma, angle-closure glaucoma, secondary glaucoma, and childhood glaucoma), Stargardt's disease, sickle cell retinopathy, radiation retinopathy, optic neuropathy, retinal detachment, retinal degeneration, uveitis, dry eye disease, congenital fibrosis of the extraocular muscles (CFEOM), and corneal fibrosis.

In some embodiments, the ocular fibrotic pathology is proliferative vitreoretinopathy (“PVR”).

In some embodiments, the ocular fibrotic pathology is selected from: opacification and fibrosis of the posterior capsule of the lens following eye surgery, fibrosis following glaucoma filtration surgery, fibrosis following a wound or trauma, conjunctival fibrosis or subconjunctival fibrosis, fibrosis of the ocular muscles, Graves disease, fibrosis following wound healing of the skin around the eye and face, fibrosis of the surface of the eye with pterygium or pingueculae, fibrosis due to choroidal neovascularization and angiogenesis, fibrosis following a corneal wound, fibrosis following corneal laser surgery, fibrosis following refractive surgery, and fibrosis following a corneal transplant.

In some embodiments, the ocular fibrotic pathology is selected from: cataract, ocular melanoma, conjunctival melanoma, retinoblastoma, optic neuritis, ocular cicatricial pemphigoid, ocular surface squamous neoplasia, keratoconus, corneal dystrophies, anterior basement membrane dystrophy, Salzmann's nodular degeneration, corneal diseases, scleritis, Fuch's endothelial corneal dystrophy, ocular lymphoma, myopia, strabismus, nystagmus, corneal haze, corneal scarring, corneal neovascularization, lacrimal gland tumors, primary open angle glaucoma, juvenile glaucoma, angle closure glaucoma, exfoliation glaucoma, and optic nerve disorders.

In some embodiments, the administering of the compound comprises administering the compound to the subject by an ocular route.

In some embodiments, the ocular route is selected from: intraocular, periocular, subtenon, retrobulbar, intravitreal, intraocular, intracameral, subconjunctival, subtenon, intracorneal, intrastromal, trans-scleral, and suprachoroidal route.

In some embodiments, the ocular route comprises a local injection into or about cornea, choroid, retina, vitreous, anterior chamber, sclera, suprachoroidal space, uvea, orbit, eyelid, conjunctiva, or iris.

In some embodiments, the administering of the compound comprises administering the compound in a pharmaceutical formulation selected from: eye-drops, eye ointment, and eye emulsion.

In another general aspect, the present disclosure provides a method of inhibiting epithelial to mesenchymal transition (EMT) in a retinal pigment epithelial (RPE) cell, the method comprising contacting the cell with an effective amount of a dopamine receptor D2 (DRD2) antagonist, or a pharmaceutically acceptable salt thereof.

In another general aspect, the present disclosure provides a method of inhibiting migration or proliferation of a retinal pigment epithelial (RPE) cell, the method comprising contacting the cell with an effective amount of a dopamine receptor D2 (DRD2) antagonist, or a pharmaceutically acceptable salt thereof.

In another general aspect, the present disclosure provides a method of inhibiting expression of a profibrotic gene in a retinal pigment epithelial (RPE) cell, the method comprising contacting the cell with an effective amount of a dopamine receptor D2 (DRD2) antagonist, or a pharmaceutically acceptable salt thereof.

In some embodiments, profibrotic gene is selected from Acta2 (α-smooth muscle actin, αSMA), Ctgf (Connective tissue growth factor), Fn1 (Fibronectin), Col1a1 (Collagen I), Col1a2 (Collagen II), and Col3a1 (Collagen III), or any combination thereof.

In another general aspect, the present disclosure provides a method of inhibiting extra-cellular matrix production and deposition by a retinal pigment epithelial (RPE) cell, the method comprising contacting the cell with an effective amount of a dopamine receptor D2 (DRD2) antagonist, or a pharmaceutically acceptable salt thereof.

In another general aspect, the present disclosure provides a method of enhancing extra-cellular matrix degradation by a retinal pigment epithelial (RPE) cell, the method comprising contacting the cell with an effective amount of a dopamine receptor D2 (DRD2) antagonist, or a pharmaceutically acceptable salt thereof.

In some embodiments, the contacting is carried out in vitro, in vivo, or ex vivo.

In some embodiments, the dopamine receptor D2 (DRD2) antagonist is selected from loxapine, clozapine, amoxapine, olanzapine, N-desmethyl olanzapine, quetiapine, N-desmethyl clozapine, 8-OH-loxapine, pizotifen, asenapine, and blonanserin, or a pharmaceutically acceptable salt thereof.

In some embodiments, the dopamine receptor D2 (DRD2) antagonist is selected from domperidone, pimozide, and sertindole, or a pharmaceutically acceptable salt thereof.

In some embodiments, the dopamine receptor D2 (DRD2) antagonist is selected from prochlorperazine, trifluoperizine, and perphenazine, or a pharmaceutically acceptable salt thereof.

In some embodiments, the dopamine receptor D2 (DRD2) antagonist is selected from eticlopride, sulpiride, remoxipride, amisulpride, and raclopride, or a pharmaceutically acceptable salt thereof.

In some embodiments, the dopamine receptor D2 (DRD2) antagonist is selected from methotrexate, spiperone, fluspirilene, penfluridol, droperidol, timiperone, benperidol, aripiprazole, and lurasidone, or a pharmaceutically acceptable salt thereof.

In another general aspect, the present disclosure provides a compound of Formula (I):

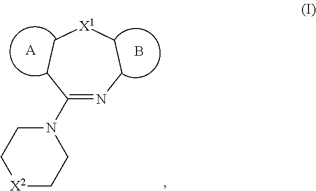

or a pharmaceutically acceptable salt thereof, wherein:
X¹ is selected from O, S, CH₂, and NR^{N};
X² is selected from O, S, and NR^{N};
R^{N} is selected from H, C₁₋₃ alkyl, and C₁₋₃ haloalkyl;
ring A is selected from phenyl and 5-6-membered heteroaryl which is optionally substituted with 1, 2, or 3 substituents independently selected from R^{A}.
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
provided that at least one of ring A and ring B is 5-6-membered heteroaryl.

In some embodiments, the compound of Formula (I) has formula:

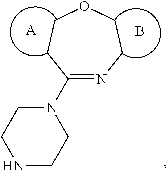

or a pharmaceutically acceptable salt thereof.

In some embodiments, the compound of Formula (I) has formula:

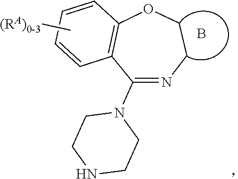

or a pharmaceutically acceptable salt thereof, wherein ring B is 5-6-membered heteroaryl which is optionally substituted with 1, 2, or 3 substituents independently selected from R^{B}.

In some embodiments, ring B is 5-membered heteroaryl which is optionally substituted with R^{B}.

In some embodiments, the 5-membered heteroaryl is selected from thienyl, furyl, and pyrrolyl.

In some embodiments, the 5-membered heteroaryl is thienyl.

In some embodiments, ring B is 6-membered heteroaryl which is optionally substituted with R^{B}.

In some embodiments, the 6-membered heteroaryl is selected from pyridinyl, pyrazinyl, and pyrimidinyl.

In some embodiments, the 6-membered heteroaryl is pyridinyl.

In some embodiments, the compound of Formula (I) has formula:

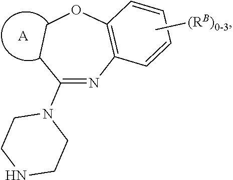

or a pharmaceutically acceptable salt thereof, wherein ring A is 5-6-membered heteroaryl which is optionally substituted with 1, 2, or 3 substituents independently selected from R^{A}.

In some embodiments, ring A is 5-membered heteroaryl which is optionally substituted with R^{A}.

In some embodiments, the 5-membered heteroaryl is selected from thienyl, furyl, and pyrrolyl.

In some embodiments, the 5-membered heteroaryl is thienyl.

In some embodiments, ring B is 6-membered heteroaryl which is optionally substituted with R^{B}.

In some embodiments, the 6-membered heteroaryl is selected from pyridinyl, pyrazinyl, and pyrimidinyl.

In some embodiments, the 6-membered heteroaryl is pyridinyl.

In some embodiments, R^{A} is selected from OH, C₁₋₃ alkoxy, SH, NH₂, C₁₋₃ alkylamino, and di(C₁₋₃ alkyl)amino.

In some embodiments, R^{A} is selected from OH, C₁₋₃ alkoxy, and NH₂.

In some embodiments, R^{B} is selected from OH, C₁₋₃ alkoxy, SH, NH₂, C₁₋₃ alkylamino, and di(C₁₋₃ alkyl)amino.

In some embodiments, R^{B} is selected from OH, C₁₋₃ alkoxy, and NH₂.

In some embodiments, the compound of Formula (I) is selected from:

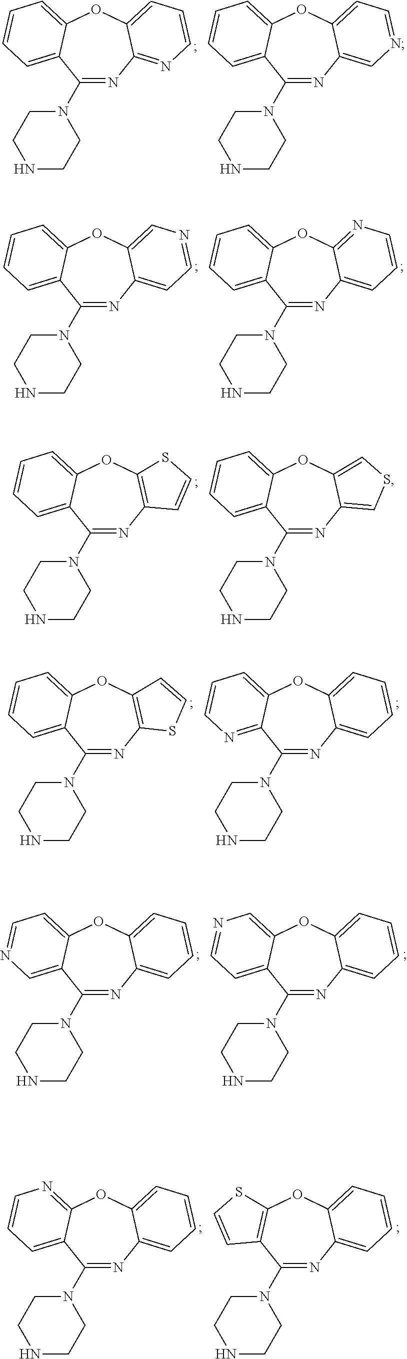

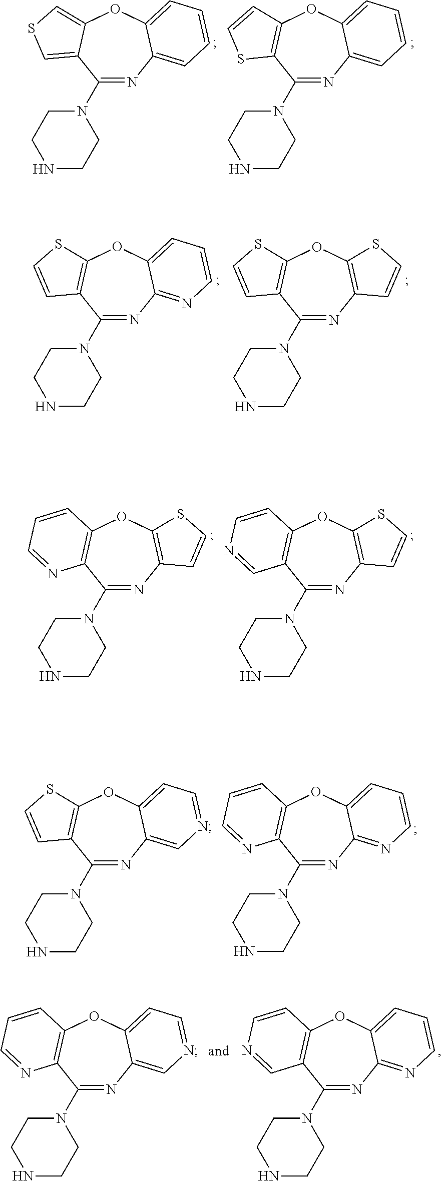

or a pharmaceutically acceptable salt thereof.

In some embodiments, the present disclosure provides a compound of Formula (II):

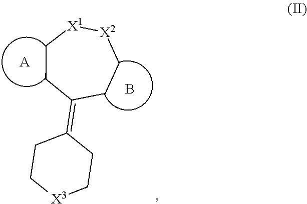

or a pharmaceutically acceptable salt thereof, wherein:
X¹ is selected from O, S, CH₂, and NR^{N};
X² is selected from O, S, CH₂, and NR^{N};
X³ is selected from O, S, and NR^{N};
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino.

In some embodiments, the compound of Formula (II) has formula:

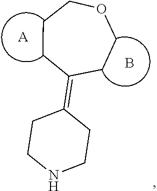

or a pharmaceutically acceptable salt thereof.

In some embodiments, the compound of Formula (II) has formula:

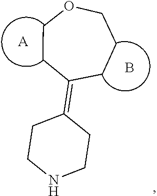

or a pharmaceutically acceptable salt thereof.

In some embodiments, the compound of Formula (II) has formula:

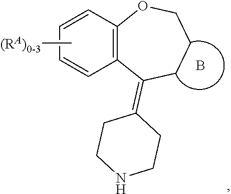

or a pharmaceutically acceptable salt thereof, wherein B is 5-6-membered heteroaryl which is optionally substituted with 1, 2, or 3 substituents independently selected from R^{B}.

In some embodiments, the compound of Formula (II) has formula:

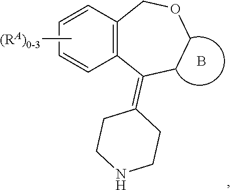

or a pharmaceutically acceptable salt thereof, B is 5-6-membered heteroaryl which is optionally substituted with 1, 2, or 3 substituents independently selected from R^{B}.

In some embodiments, ring B is 5-membered heteroaryl which is optionally substituted with R^{B}.

In some embodiments, the 5-membered heteroaryl is selected from thienyl, furyl, and pyrrolyl.

In some embodiments, the 5-membered heteroaryl is thienyl.

In some embodiments, ring B is 6-membered heteroaryl which is optionally substituted with R^{B}.

In some embodiments, the 6-membered heteroaryl is selected from pyridinyl, pyrazinyl, and pyrimidinyl.

In some embodiments, the 6-membered heteroaryl is pyridinyl.

In some embodiments, the compound of Formula (II) has formula:

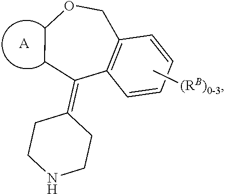

or a pharmaceutically acceptable salt thereof, wherein ring A is 5-6-membered heteroaryl which is optionally substituted with 1, 2, or 3 substituents independently selected from R^{A}.

In some embodiments, the compound of Formula (II) has formula:

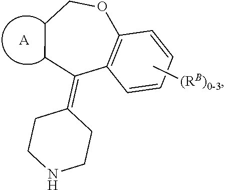

or a pharmaceutically acceptable salt thereof, wherein ring A is 5-6-membered heteroaryl which is optionally substituted with 1, 2, or 3 substituents independently selected from R^{A}.

In some embodiments, ring A is 5-membered heteroaryl which is optionally substituted with R^{A}.

In some embodiments, the 5-membered heteroaryl is selected from thienyl, furyl, and pyrrolyl.

In some embodiments, the 5-membered heteroaryl is thienyl.

In some embodiments, ring B is 6-membered heteroaryl which is optionally substituted with R^{B}.

In some embodiments, the 6-membered heteroaryl is selected from pyridinyl, pyrazinyl, and pyrimidinyl.

In some embodiments, the 6-membered heteroaryl is pyridinyl.

In some embodiments, R^{A} is selected from OH, C₁₋₃ alkoxy, SH, NH₂, C₁₋₃ alkylamino, and di(C₁₋₃ alkyl)amino.

In some embodiments, R^{A} is selected from OH, C₁₋₃ alkoxy, and NH₂.

In some embodiments, R^{B} is selected from OH, C₁₋₃ alkoxy, SH, NH₂, C₁₋₃ alkylamino, and di(C₁₋₃ alkyl)amino.

In some embodiments, R^{B} is selected from OH, C₁₋₃ alkoxy, and NH₂.

In some embodiments, the compound of Formula (II) is selected from:

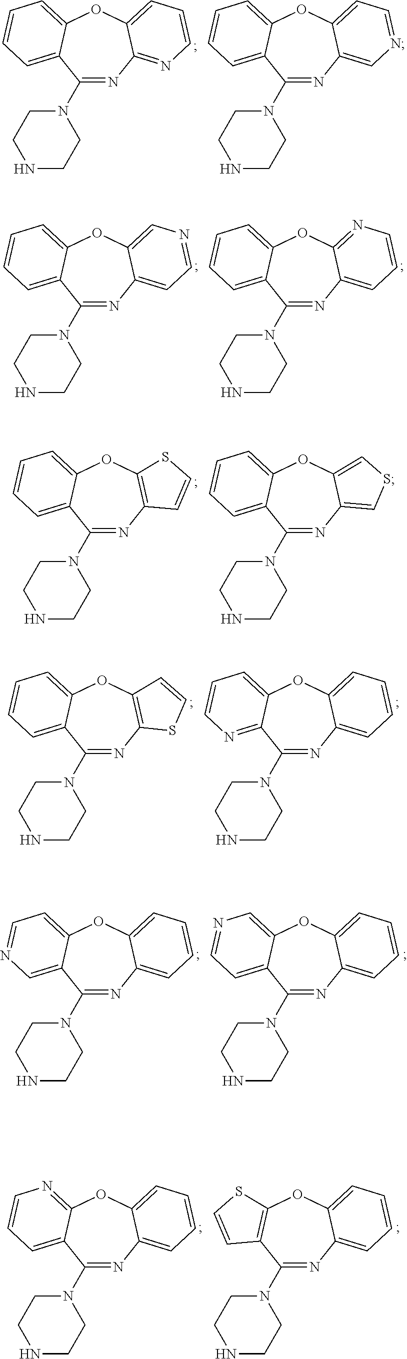

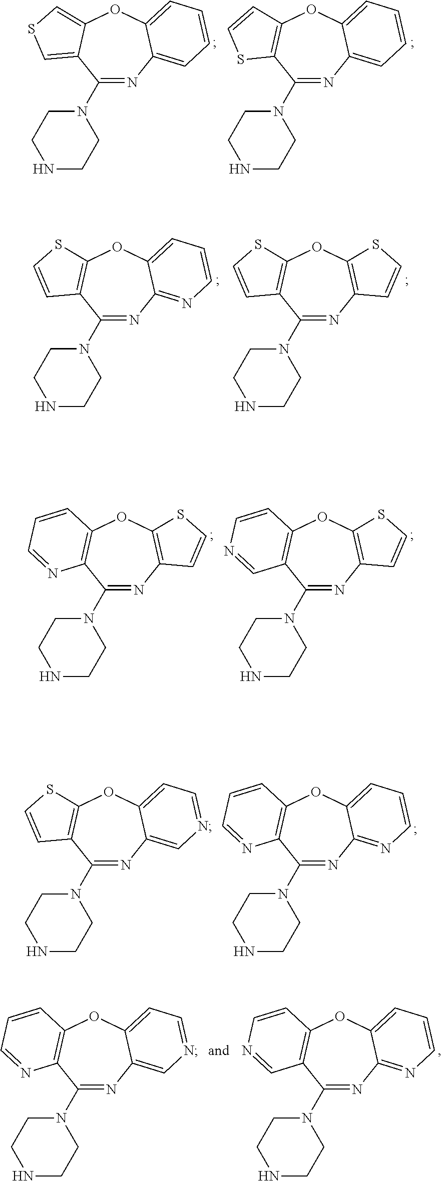

or a pharmaceutically acceptable salt thereof.

In some embodiments, the present disclosure provides a pharmaceutical composition comprising as described herein, or a pharmaceutically acceptable salt thereof, and a pharmaceutically acceptable carrier.

In some embodiments, a method of treating or preventing an ocular fibrotic pathology, the method comprising administering to a subject in need thereof a therapeutically effective amount of a compound as described herein, or a pharmaceutically acceptable salt thereof.

In some embodiments, the ocular fibrotic pathology is selected from: proliferative vitreoretinopathy (PVR), diabetic retinopathy, ischemic retinopathy, age-related macular degeneration (ARMD), dry ARMD, neovascular ARMD, keratitis, pterygia, pingueculae, retinopathy of prematurity, glaucoma (including neovascular glaucoma, open-angle glaucoma, angle-closure glaucoma, secondary glaucoma, and childhood glaucoma), Stargardt's disease, sickle cell retinopathy, radiation retinopathy, optic neuropathy, retinal detachment, retinal degeneration, uveitis, dry eye disease, congenital fibrosis of the extraocular muscles (CFEOM), and corneal fibrosis.

In some embodiments, the ocular fibrotic pathology is proliferative vitreoretinopathy (“PVR”).

In some embodiments, the ocular fibrotic pathology is selected from: opacification and fibrosis of the posterior capsule of the lens following eye surgery, fibrosis following glaucoma filtration surgery, fibrosis following a wound or trauma, conjunctival fibrosis or subconjunctival fibrosis, fibrosis of the ocular muscles, Graves disease, fibrosis following wound healing of the skin around the eye and face, fibrosis of the surface of the eye with pterygium or pingueculae, fibrosis due to choroidal neovascularization and angiogenesis, fibrosis following a corneal wound, fibrosis following corneal laser surgery, fibrosis following refractive surgery, and fibrosis following a corneal transplant.

In some embodiments, the ocular fibrotic pathology is selected from: cataract, ocular melanoma, conjunctival melanoma, retinoblastoma, optic neuritis, ocular cicatricial pemphigoid, ocular surface squamous neoplasia, keratoconus, corneal dystrophies, anterior basement membrane dystrophy, Salzmann's nodular degeneration, corneal diseases, scleritis, Fuch's endothelial corneal dystrophy, ocular lymphoma, myopia, strabismus, nystagmus, corneal haze, corneal scarring, corneal neovascularization, lacrimal gland tumors, primary open angle glaucoma, juvenile glaucoma, angle closure glaucoma, exfoliation glaucoma, and optic nerve disorders.

In some embodiments, the administering of the compound comprises administering the compound to the subject by an ocular route.

In some embodiments, the ocular route is selected from: intravitreal, intraocular, intracameral, subconjunctival, subtenon, intracorneal, intrastromal, trans-scleral, and suprachoroidal route.

In some embodiments, the ocular route comprises a local injection into or about cornea, choroid, retina, vitreous, uvea, orbit, eyelid, conjunctiva, or iris.

In some embodiments, the administering of the compound comprises administering the compound in a pharmaceutical formulation selected from: eye-drops, eye ointment, and eye emulsion.

In some embodiments, the present disclosure provides a method of inhibiting epithelial to mesenchymal transition (EMT) in a retinal pigment epithelial (RPE) cell, the method comprising contacting the cell with an effective amount of a compound as described herein, or a pharmaceutically acceptable salt thereof.

In some embodiments, the present disclosure provides a method of inhibiting migration or proliferation of a retinal pigment epithelial (RPE) cell, the method comprising contacting the cell with an effective amount of a compound as described herein, or a pharmaceutically acceptable salt thereof.

In some embodiments, the present disclosure provides a method of inhibiting expression of a profibrotic gene in a retinal pigment epithelial (RPE) cell, the method comprising contacting the cell with an effective amount of a compound as described herein, or a pharmaceutically acceptable salt thereof.

In some embodiments, profibrotic gene is selected from Acta2 (α-smooth muscle actin, αSMA), Ctgf (Connective tissue growth factor), Fn1 (Fibronectin), Col1a1 (Collagen I), Col1a2 (Collagen II), and Col3a1 (Collagen III), or any combination thereof.

In some embodiments, the present disclosure provides a method of inhibiting extra-cellular matrix production and deposition by a retinal pigment epithelial (RPE) cell, the method comprising contacting the cell with an effective amount of a compound as described herein, or a pharmaceutically acceptable salt thereof.

In some embodiments, the present disclosure provides a method of enhancing extra-cellular matrix degradation by a retinal pigment epithelial (RPE) cell, the method comprising contacting the cell with an effective amount of a compound as described herein, or a pharmaceutically acceptable salt thereof.

In some embodiments, the contacting is carried out in vitro, in vivo, or ex vivo.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1A Dopamine receptor expression and dopamine synthesis in culture RPE cells. Expression of dopamine receptor family in cultured ARPE-19 cells treated±TGFβ for 24 hours. RNA expression is shown, relative to GAPDH, normalized to the mean expression of DRD2 in n=6 independent experiments. Results are expressed as the mean±s.e.m. Y-axis is logarithmic.

FIG. 1B In vitro dopamine production by cultured ARPE-19 cells treated±TGFβ for 24 hours. n=4 independent experiments. Comparison made by paired t-test, * p<0.05. Results are expressed as the mean±s.e.m.

FIG. 2A D2 dopamine receptor inhibition promote antifibrotic gene expression. Coupling of D2 dopamine receptor to Gαᵢ is activated by pramipexole (PMX), a D2 dopamine receptor agonist, and inhibited by loxapine (LOX), a D2 dopamine receptor antagonist.

FIG. 2B Effect of agonists and antagonists of the D2 dopamine receptor on profibrotic gene expression in cultured ARPE-19 cells. ARPE-19 cells were treated±TGFβ, pramipexole (PMX), and loxapine (LOX) for 24 hours. n=3 independent experiments. Comparison made by ANOVA, ** p<0.01, *** p<0.001, **** p<0.0001. Results are expressed as the mean±s.e.m.

FIG. 3A Antifibrotic effect of D2 receptor antagonist loxapine. Wound migration assay. ARPE-19 cells were plated to confluence prior to the formation of a “wound.” Cells were then treated±TGFβ, ±10 μM loxapine and the wound area was imaged and quantified every 24 hours. Shown are representative images from the 0- and 72-hour timepoints for each of the conditions. n=3 independent experiments.

FIG. 3B Cellular proliferation of ARPE-19 cells treated±2% FBS, 10p M loxapine for 4 days. Proliferation was assessed by Cell-titer Fluor. n=3 independent experiments. Comparison made by ANOVA, ** p<0.01, *** p<0.001. Results are expressed as the mean+s.e.m.

FIG. 3C Live/Dead assessment of ARPE-19 cells treated for 4 days±2% FBS, ±10 μM loxapine. Data are expressed as the number of individual cells within the field of view identified as Live (green), or dead (red). Show are representative images after 4 days in culture for each of the condition. n=3 independent experiments. Comparison made by ANOVA, ** p<0.01, *** p<0.001. Results are expressed as the mean±s.e.m.

FIG. 3D Fibronectin deposition of ARPE-19 cells cultured for 4 days±TGFβ, ±10 μM loxapine. Cells were fixed and immunostained for fibronectin and counterstained with DAPI. Show are representative images for each of the condition. Fibronectin intensity, relative to DAPI cell counts was quantified using automated imaging software (Gen5). n=3 independent experiments. Comparison made by ANOVA, **** p<0.0001. Results are expressed as the mean±s.e.m.

FIG. 4A shows results of fibronectin deposition assay for compounds loxapine, olanzapine, N-desmethyl clozapine, including their chemical structures.

FIG. 4B shows results of fibronectin deposition assay for compounds clozapine, N-desmethyl olanzapine, and 8-OH-loxapine, including their chemical structures.

FIG. 4C shows results of fibronectin deposition assay for compounds amoxapine, quetiapine, and pizotifen, including their chemical structures.

FIG. 4D shows results of fibronectin deposition assay for compounds asenapine and blonanserin, including their chemical structures.

FIG. 5A shows results of fibronectin deposition assay for compounds domperidone, pimozide, and sertindole, including their chemical structures.

FIG. 5B shows results of fibronectin deposition assay for compounds prochlorperazine, trifluoperizine, and perphenazine, including their chemical structures.

FIG. 5C shows results of fibronectin deposition assay for compounds eticlopride and sulpiride, including their chemical structures.

FIG. 5D shows results of fibronectin deposition assay for compounds remoxipride, amisulpride, and raclopride, including their chemical structures.

FIG. 6A shows results of fibronectin deposition assay for solnitropine, including its chemical structure.

FIG. 6B shows results of a fibronectin deposition assay for solupine, including its chemical structure.

FIG. 7A Effect of antagonists of the D2 dopamine receptor (loxapine, solnitropine, and solupine) on profibrotic COL1A1 gene expression in cultured ARPE-19 cells. ARPE-19 cells were treated±TGFβ, Loxapine (10 μM), Solnitropine (10 μM), or Solupine (10 μM) (for 24 hours prior to RNA isolation and subsequent qPCR analysis.

FIG. 7B Effect of antagonists of the D2 dopamine receptor (loxapine, solnitropine, and solupine) on profibrotic FN1 gene expression in cultured ARPE-19 cells. ARPE-19 cells were treated±TGFβ, Loxapine (10 μM), Solnitropine (10 μM), or Solupine (10 μM) (for 24 hours prior to RNA isolation and subsequent qPCR analysis.

FIG. 8 shows differential solubility of D2 dopamine receptor antagonists oxapine, solnitropine, and solupine in phosphate buffered saline, determined by solution turbidity measured by the absorbance of 620 nm light. Elevated absorbance indicates precipitation and insolubility.

FIG. 9A shows results of fibronectin deposition assay for compounds methotrexate, spiperone, and fluspirilene, including their chemical structures.

FIG. 9B shows results of fibronectin deposition assay for compounds penfluridol, droperidol, and timiperone, including their chemical structures.

FIG. 9C shows results of fibronectin deposition assay for compounds benperidol and lurasidone, including their chemical structures.

FIG. 10A contains line plot showing effect of methotrexate, solupine, and sertindone on FGF-β stimulated proliferation.

FIG. 10B contains line plot showing effect of penfluridol, pimozide, and fluspirilene on FGF-β stimulated proliferation.

FIG. 11 contains line plot showing effect of methotrexate and solupine on PDGF-CC stimulated proliferation.

FIG. 12 is a line plot showing that Solupine stock is functionally stable for at least 90 days under these conditions

FIG. 13 is a bar graph showing solupine does not block fibronectin deposition by conjunctival fibroblasts.

FIG. 14 is a bar graph showing mRNA expression of DRD1 and DRD2 in corneal epithelia.

FIG. 15 is a bar graph showing that solupine blocks fibronectin deposition in ARPE-19 cells stimulated with a cocktail of profibrotic ligands.

FIG. 16 is a line plot showing that aripiprazole block fibronectin deposition.

FIG. 17A is a bar graph showing toxicology scoring of mouse eye injected with solupine (PBS).

FIG. 17B is a bar graph showing toxicology scoring of mouse eye injected with solupine (10 μg/0.05 mL solupine).

FIG. 17C is a bar graph showing toxicology scoring of mouse eye injected with solupine (30 μg/0.05 mL solupine).

### DETAILED DESCRIPTION

Retinal pigment epithelial (RPE) cells play an important role in maintaining the structural and functional health of the retinal, macular, and associated vasculature. RPE cells form a monocellular layer immediately behind the retina and play an essential role in light absorption, barrier function, and fluid/ion transport. Dysfunction of these cells plays a role in multiple ocular diseases including age-related macular degeneration and proliferative vitreoretinopathy. Aging, inflammation, and acute injury (e.g., retinal injury due to retinal detachment) can all lead to epithelial to mesenchymal transition (EMT) in RPE cells (trans-differentiation into fibroblast-like mesenchymal cells), stimulating cellular proliferation, migration, contraction, and deposition of extracellular matrix (ECM, e.g., type I collagen and fibronectin); all of which contribute to ocular fibrosis and lead to associated diseases (e.g., PVR).

Accordingly, in some embodiments, the present disclosure provides a method of inhibiting epithelial to mesenchymal transition (EMT) in a retinal pigment epithelial (RPE) cell, the method comprising contacting the cell with an effective amount of a compound as disclosed herein, or a pharmaceutically acceptable salt thereof. In some embodiments, the contacting is carried out in vitro, in vivo, or ex vivo. Hence, in some embodiments, the present disclosure provides a method of inhibiting epithelial to mesenchymal transition (EMT) in a retinal pigment epithelial (RPE) cell of a subject, the method comprising administering to the subject a therapeutically effective amount of a compound as disclosed herein, or a pharmaceutically acceptable salt thereof.

Without being bound by any theory, it is believed that G protein coupled receptor (“GPCR”) signaling is involved in regulation of profibrotic phenotypes of RPE cells (as well as mesenchymal cells from many organs prone to tissue fibrosis). Generally, GPCRs are divided into families by the ligands which endogenously stimulate their signaling cascades. Concerning dopamine receptors, there are five: dopamine receptors D1 (DRD1) and D5 (DRD5) which are considered “D1-like”, and dopamine receptors D2 (DRD2), D3 (DRD3), and D4 (DRD4) being “D2-like.” Further, GPCRs are linked to effector proteins from four main classes of G-proteins (e.g., Gα_{12/13}, Gα_{q/11}, Gα_{i/o}, or Gαₛ). Following agonist stimulation, members of the D1-like couple to Gαₛ, elevating intracellular cAMP which leads to inhibition of downstream transcriptional programs such as YAP/TAZ and MRTFA/B and overall anti-fibrotic response. In contrast, following agonist stimulation, members of the D2-like couple to and activate Gαᵢ, leading to repression of intracellular cAMP and a profibrotic response promoting profibrotic transcriptional programs. As the results presented in this disclosure demonstrate, selective D2 dopamine receptor antagonists inhibit profibrotic gene expression.

Without being bound by any theory or speculation, it is believed that inactivation (antagonism) of a G alpha i (Gαᵢ) protein coupled receptor (e.g., DRD2 as described herein) in RPE cells blocks (or inhibits) EMT in these cells (e.g., antagonizing GαᵢPCR blocks expression of genes associated with EMT), thereby inhibiting or preventing proliferation and/or migration of RPE cells, and inhibiting or preventing secretion of components of extracellular matrix. Hence, antagonizing GαᵢPCR in RPE cells allows to maintain epithelial nature of these cells and to maintain RPE cell function. For example, antagonizing GαᵢPCR in RPE cells results in inhibition or prevention of expression of profibrotic genes, such as Acta2 (αSMA), Ctgf (Connective tissue growth factor), Fn1 (Fibronectin), Col1a1 (Collagen I), Col1a2 (Collagen II), and Col3a1 (Collagen III).

Accordingly, in some embodiments, the present disclosure provides a method of antagonizing a Gαᵢ protein coupled receptor in a retinal pigment epithelial (RPE) cell, the method comprising contacting the cell with an effective amount of a compound as disclosed herein, or a pharmaceutically acceptable salt thereof. In some embodiments, the contacting is carried out in vitro, in vivo, or ex vivo. Hence, in some embodiments, the present disclosure provides a method of antagonizing a Gαᵢ protein coupled receptor in a retinal pigment epithelial (RPE) cell of a subject, the method comprising administering to the subject a therapeutically effective amount of a compound as disclosed herein, or a pharmaceutically acceptable salt thereof. In some embodiments, the antagonizing is selective (exclusive), e.g., the antagonizing is 100-fold, 50-fold, or 10-fold selective to Gαᵢ protein coupled receptor as compared to Gα_{12/13}, Gα_{q/11}, or Gαₛ protein coupled receptor, or any combination of the aforementioned).

Accordingly, in some embodiments, the present disclosure provides a method of antagonizing a dopamine receptor D2 (DRD2) in a retinal pigment epithelial (RPE) cell, the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method of antagonizing a dopamine receptor D2 (DRD2) in a retinal pigment epithelial (RPE) cell of a subject, the method comprising administering to the subject a therapeutically effective amount of a compound disclosed herein, or a pharmaceutically acceptable salt thereof. In some embodiments, antagonizing DRD2 is selective with respect to DRD2 (e.g., the method does not substantially involve antagonizing or agonizing D1, D3, D4, or D5 receptor, or any combination of the aforementioned). For example, the antagonizing is 100-fold, 50-fold, or 10-fold selective to D2 dopamine receptor (e.g., compounds of this disclosure are selective antagonists of DRD2 and do not substantially modulate (agonize or antagonize) D1, D3, D4, or D5 receptor, or any combination of the aforementioned). In some embodiments, DRD2 antagonist also antagonizes “D2-like” receptors (DRD3 and DRD4). In some embodiments, DRD2 is preferentially expressed in a retinal pigment epithelial (RPE) cell. For example, DRD2 comprises 51%, 60%, 80%, 90%, 95%, 99%, or 100% of all dopamine receptors expressed in the RPE cell.

Accordingly, in some embodiments, the present disclosure provides a method of inhibiting expression of a profibrotic gene in a retinal pigment epithelial (RPE) cell, the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method inhibiting expression of a profibrotic gene in a retinal pigment epithelial (RPE) cell of a subject, the method comprising administering to the subject a therapeutically effective amount of a compound disclosed herein, or a pharmaceutically acceptable salt thereof. In some embodiments, the profibrotic gene is selected from Acta2 (α-smooth muscle actin, αSMA), Ctgf (Connective tissue growth factor), Fn1 (Fibronectin), Col1a1 (Collagen I), Col1a2 (Collagen II), and Col3a1 (Collagen III), or any combination thereof.

In some embodiments, the present disclosure provides a method of inhibiting proliferation of a retinal pigment epithelial (RPE) cell, the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method inhibiting proliferation of a retinal pigment epithelial (RPE) cell in a subject, the method comprising administering to the subject a therapeutically effective amount of a compound disclosed herein, or a pharmaceutically acceptable salt thereof.

In some embodiments, the present disclosure provides a method of inhibiting migration of a retinal pigment epithelial (RPE) cell (e.g., in an ocular tissue), the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method inhibiting migration of a retinal pigment epithelial (RPE) cell (e.g., in an ocular tissue) in a subject, the method comprising administering to the subject a therapeutically effective amount of a compound disclosed herein, or a pharmaceutically acceptable salt thereof.

In some embodiments, the present disclosure provides a method of inhibiting secretion of a component of extracellular matrix from a retinal pigment epithelial (RPE) cell, the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method of inhibiting secretion of a component of extracellular matrix from a retinal pigment epithelial (RPE) cell of a subject, the method comprising administering to the subject a therapeutically effective amount of a compound disclosed herein, or a pharmaceutically acceptable salt thereof. The method includes inhibiting extra-cellular matrix production and deposition by an RPE cell.

Suitable examples of components of extracellular matrix include proteins, glycosaminoglycans (mucopolysaccharides), and glycoconjugates (glycans, or polysaccharides, that are covalently linked to proteins, peptides, or lipids). Examples of glycoconjugates of the extracellular matrix include glycoproteins, proteoglycans, glycopeptides, peptidoglycans, glycolipids, glycosides, and lipopolysaccharides, or any combination of the aforementioned. Examples of glycosaminoglycans of the extracellular matrix include hyaluronic acid, chondroitin sulfate, dermatan sulfate, keratan sulfate, and heparan sulfate. Examples of proteoglycans of the extracellular matrix include aggrecan, versican, neurocan, and brevican. Examples of glycoproteins include tenascin, fibronectin, laminin, osteopontin, fibulin, and matricellar glycoproteins, or any combination of the aforementioned. Examples of proteins of the extracellular matrix include collagen (e.g., type I, II, III, IV, V, or VI), elastin, tropoeslastin, fibrillin, fibrin, fibrinogen, fibronectin, and laminin, or any combination of the aforementioned.

In some embodiments, the present disclosure provides a method of inhibiting deposition and/or accumulation of extracellular matrix in an ocular tissue (e.g., in or near an RPE cell), the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method of inhibiting deposition and/or accumulation of extracellular matrix in an ocular tissue (e.g., in or near an RPE cell) of a subject, the method comprising administering to the subject a therapeutically effective amount of a compound disclosed herein, or a pharmaceutically acceptable salt thereof. The method includes enhancing extra-cellular matrix degradation by an RPE cell.

In some embodiments, the present disclosure provides a method of reversing fiber formation and extracellular matrix accumulation in an ocular tissue. Without being bound by any theory, it is believed that agonizing GPCR and/or dopamine receptor in an RPE cell (e.g., as described herein) reverses formation of components of extracellular matrix in an ocular tissue and results in dissolution of the extracellular matrix that already accumulated in the ocular tissue.

In some embodiments, epithelial to mesenchymal transition (EMT) in RPE cells, stimulating RPE cellular proliferation and/or migration, and/or extracellular matrix (ECM) deposition (fibrosis, scarring) in an ocular tissue is induced by (or results from) trauma, recovery after surgery (e.g., cataract surgery), ocular tissue injury (e.g., open globe injury), aging, inflammation, infection (e.g., bacterial, fungal, or viral infection), intraocular pressure, genetic predisposition, co-morbidity, damage to optic nerve, tissue ischemia, retinal detachment, vascular leakage, hemorrhage, or any combination of these factors. Normally cells in the ocular tissues, such as the RPE cells, generate just the right amount of tissue to replace old tissue or repair damage. Excessive connective tissue generation (e.g., in response to trauma or injury as discussed above) results in pathological accumulation of fibrotic tissue (e.g., extracellular matrix proteins) leading to tissue thickening, fibrosis, and scarring.

In some embodiments, the present disclosure provides a method of inhibiting (or reversing) an ocular tissue fibrosis (inhibiting fibrosis in an ocular tissue). Suitable examples of ocular tissues include iris, cornea, conjunctiva, retina (including neural retina), retinal pigment epithelium, choriocapillaris, sclera, nerve fibers, ganglion cells, choroid, choroidal vessels, uvea, ciliary body, fovea, Schlemm's canal, trabecular meshwork, corneal stroma, and macula. In some embodiments, the ocular tissue fibrosis is associated with RPE cells.

In some embodiments, the present disclosure provides a method of treating or preventing an ocular fibrotic pathology (e.g., an ocular disease or condition in which fibrosis is implicated) in a subject, the method comprising administering to the subject in need thereof a therapeutically effective amount of any one of the compounds described herein, or a pharmaceutically acceptable salt thereof.

In some embodiments, the subject in need of treatment of an ocular fibrotic pathology is diagnosed with an ocular fibrotic pathology by a treating physician. In some embodiments, the subject in need of prevention of an ocular fibrotic pathology is diagnosed with an ocular tissue trauma or injury, ocular infection, ocular inflammation, increased intraocular pressure, retinal or subretinal neovascularization, genetic predisposition, co-morbidity (e.g., diabetes or another metabolic disease), damage to optic nerve, or a similar condition, by a treating physician. Suitable examples of ocular tissue injuries include a drug-induced injury (injury caused by an antibiotic or an anticancer drug), tissue injury caused by autoimmune disease, including sepsis, tissue ischemia, vascular leakage, hemorrhage, including subretinal hemorrhage, macular edema, chronic wound healing, and injury caused by an infection.

Ocular fibrosis contributes to visual loss in millions of people globally. The compounds within the present claims (e.g., DRD2 receptor antagonists) advantageously treat or prevent the ocular fibrotic pathologies and therefore prevent or decrease the visual loss.

In some embodiments, an ocular fibrotic pathology is selected from: proliferative vitreoretinopathy (“PVR”), epiretinal membrane, diabetic retinopathy, ischemic retinopathy, macular degeneration, age-related macular degeneration (“ARMD,” including dry ARMD and neovascular ARMD), keratitis, pterygia, pingueculae, retinopathy of prematurity, glaucoma (including neovascular glaucoma, open-angle glaucoma, angle-closure glaucoma, secondary glaucoma, and childhood glaucoma), Stargardt's disease, sickle cell retinopathy, radiation retinopathy, optic neuropathy, retinal detachment, retinal degeneration, uveitis, dry eye disease, congenital fibrosis of the extraocular muscles (“CFEOM”), and corneal fibrosis. In some embodiments, the ocular fibrotic pathology is selected from: opacification and fibrosis of the posterior capsule of the lens following eye surgery, fibrosis following glaucoma filtration surgery, fibrosis following a wound or trauma, conjunctival fibrosis or subconjunctival fibrosis, fibrosis of the ocular muscles, Graves disease, fibrosis following wound healing of the skin around the eye and face, fibrosis of the surface of the eye with pterygium or pingueculae, fibrosis due to choroidal neovascularization and angiogenesis, fibrosis following a corneal wound, fibrosis following corneal laser surgery, fibrosis following refractive surgery, and fibrosis following a corneal transplant.

In some embodiments, the ocular fibrotic pathology is selected from: cataract, ocular melanoma, conjunctival melanoma, retinoblastoma, optic neuritis, ocular cicatricial pemphigoid, ocular surface squamous neoplasia, keratoconus, corneal dystrophies, anterior basement membrane dystrophy, Salzmann's nodular degeneration, corneal diseases, scleritis, Fuch's endothelial corneal dystrophy, ocular lymphoma, myopia, strabismus, nystagmus, corneal haze, corneal scarring, corneal neovascularization, lacrimal gland tumors, primary open angle glaucoma, juvenile glaucoma, angle closure glaucoma, exfoliation glaucoma, and optic nerve disorders.

Common symptoms of the aforementioned ocular fibrotic pathologies include loss of vision, blindness, mechanical disruption of the visual axis, opacification and decreased vision, or an otherwise impairment of visual function. In some embodiments, the present disclosure provides a method of reducing or ameliorating these symptoms. That is, in some embodiments, the present disclosure provides a method of increasing vision, maintaining of the visual axis in the eye, and preventing blindness.

### Therapeutic Compounds

In some embodiments, a compound that can be used in any one of the methods described here is an antagonist of a G alpha i (Gαᵢ) protein coupled receptor. In such embodiments, the compound is a selective antagonist of a Gαᵢ receptor (e.g., the compound is 100-fold, 50-fold, or 10-fold selective to Gαᵢ protein coupled receptor as compared to Gα_{12/13}, Gα_{q/11} or Gαₛ protein coupled receptor, or any combination of the aforementioned (the compound does not substantially modulate any of the aforementioned)).

In some embodiments, a compound of the present disclosure is a dopamine receptor D2 antagonist. E.g., the antagonist of a Gαᵢ protein coupled receptor antagonizes DRD2 receptor. In some embodiments, the compound is a selective antagonist of a dopamine receptor D2 (e.g., the compound is 100-fold, 50-fold, or 10-fold selective and/or specific to antagonizing D2 dopamine receptor as compared to modulating (agonizing or antagonizing) D1, D3, D4, or D5 receptor, or any combination of the aforementioned). In some embodiments, the receptor antagonist compound of this disclosure is a monoclonal or polyclonal antibody that is specific to dopamine receptor D2 and specifically antagonizes the receptor.

In some embodiments, the compound of the present disclosure (e.g., a D2 dopamine receptor antagonist) is hydrophilic. In such embodiments, the structure of the compound contains hydrogen bond donor (HBD) atoms that are capable of forming hydrogen bonds with molecules of water and with the amino acids within the active site of the Gi protein coupled receptor (e.g., D2 receptor). In some embodiments, the molecule of the receptor antagonist contains at least 2, 3, 4, 5, or 6 HBD atoms (e.g., heteroatoms such as O, N or S). In some embodiments, the molecule of the receptor antagonist contains at least one hydroxyl group (e.g., 1, 2, 3, 4, 5, or 6 hydroxyl groups). In some embodiments, the molecule of the receptor antagonist contains amino groups (e.g., 1, 2, 3, 4, 5, or 6 amino groups).

In some embodiments, the compound does not penetrate the blood brain barrier or only an insignificant amount of the receptor antagonist penetrates the blood brain barrier after the receptor antagonist is administered to a subject (e.g., not more than about 0.1 wt. %, about 1 wt. %, about 5 wt. %, about 10 wt. %, or about 20 wt. % of the amount of the compound administered to the subject penetrates the blood brain barrier). In some embodiments, the compound (e.g., a dopamine receptor antagonist) is ineffective or only weakly effective in treating central nervous system (CNS) disorders.

In some embodiments, the compound is a small molecule, e.g., about 2000 daltons or less (e.g., from about 300 to about 1200, from about 300 to about 1000, from about 300 to about 800, and/or from about 300 to about 600 daltons). In some embodiments, the compound is a biomolecule. Typically, biomolecules are organic molecules having a molecular weight of 200 daltons or more produced by living organisms or cells, including large polymeric molecules such as polypeptides, proteins, glycoproteins, polysaccharides, polynucleotides and nucleic acids. In some embodiments, the compound is an antibody, a hormone, a transmembrane protein, a growth factor, or an enzyme.

In some embodiments, the compound is selected from loxapine, clozapine, amoxapine, olanzapine, N-desmethyl olanzapine, quetiapine, N-desmethyl clozapine, 8-OH-loxapine, pizotifen, asenapine, blonanserin, domperidone, pimozide, sertindole, eticlopride, prochlorperazine, trifluoperizine, perphenazine, remoxipride, amisulpride, raclopride, and sulpiride, or a pharmaceutically acceptable salt thereof.

In some embodiments, the compound is selected from loxapine, clozapine, amoxapine, olanzapine, N-desmethyl olanzapine, quetiapine, N-desmethyl clozapine, 8-OH-loxapine, pizotifen, asenapine, and blonanserin, or a pharmaceutically acceptable salt thereof.

In some embodiments, the compound is selected from domperidone, pimozide, and sertindole, or a pharmaceutically acceptable salt thereof.

In some embodiments, the compound is selected from prochlorperazine, trifluoperizine, and perphenazine, or a pharmaceutically acceptable salt thereof.

In some embodiments, the compound is selected from eticlopride, sulpiride, remoxipride, amisulpride, and raclopride, or a pharmaceutically acceptable salt thereof.

In some embodiments, the compound is selected from methotrexate, spiperone, fluspirilene, penfluridol, droperidol, timiperone, benperidol, aripiprazole, and lurasidone, or a pharmaceutically acceptable salt thereof.

### Compound of Formula (I)

In some embodiments, the compound is a compound of Formula (I):

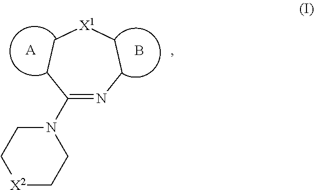

or a pharmaceutically acceptable salt thereof, wherein:
X¹ is selected from O, S, CH₂, and NR^{N};
X² is selected from O, S, and NR^{N};
R^{N} is selected from H, C₁₋₃ alkyl, and C₁₋₃ haloalkyl;
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino.

In some embodiments, at least one of ring A and ring B is 5-6-membered heteroaryl.

In some embodiments, X¹ is O. In some embodiments, X¹ is S. In some embodiments, X¹ is CH₂. In some embodiments, X¹ is NR^{N}. In some embodiments, X¹ is NH.

In some embodiments, X² is O. In some embodiments, X² is S. In some embodiments, X² is NR^{N}. In some embodiments, X² is NH.

In some embodiments X¹ is O and X² is O. In some embodiments X¹ is O and X² is S. In some embodiments X¹ is S and X² is O. In some embodiments X¹ is S and X² is S. In some embodiments X¹ is NH and X² is O. In some embodiments X¹ is NH and X² is NH.

In some embodiments, the compound of Formula (I) has formula:

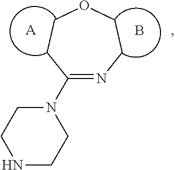

or a pharmaceutically acceptable salt thereof.

In some embodiments, ring A is phenyl, optionally substituted with 1, 2, or 3 substituents independently selected from R^{A}. In some embodiments, ring A is phenyl, optionally substituted with R^{A}. In some embodiments, ring A is phenyl.

In some embodiments, ring A is 5-6-membered heteroaryl which is optionally substituted with 1, 2, or 3 substituents independently selected from R^{A}. In some embodiments, ring A is 5-membered heteroaryl which is optionally substituted with 1, 2, or 3 substituents independently selected from R^{A}. In some embodiments, ring A is 6-membered heteroaryl which is optionally substituted with 1, 2, or 3 substituents independently selected from R^{A}. In some embodiments, ring A is 5-6-membered heteroaryl which is optionally substituted with R^{A}. In some embodiments, ring A is 5-membered heteroaryl which is optionally substituted with R^{A}. In some embodiments, ring A is 6-membered heteroaryl which is optionally substituted with R^{A}. In some embodiments, ring A is 5-6-membered heteroaryl. In some embodiments, ring A is 5-membered heteroaryl. In some embodiments, ring A is 6-membered heteroaryl. In some aspects of the foregoing embodiments, the heteroaryl is selected from thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. In other aspects of the foregoing embodiments, the heteroaryl is selected from pyridinyl (e.g., 2-pyridinyl, 3-pyridinyl, or 4-pyridinyl), pyrazinyl, pyrimidinyl, triazinyl, and pyridazinyl.

In some embodiments, ring A is selected from thienyl and pyridinyl, each of which is optionally substituted with 1, 2, or 3 independently selected R^{A}. In some embodiments, ring A is thienyl, optionally substituted with R^{A}. In some embodiments, ring A is pyridinyl, optionally substituted with R^{A}. In some embodiments, ring A is thienyl. In some embodiments, ring A is pyridinyl.

In some embodiments, ring B is phenyl, optionally substituted with 1, 2, or 3 substituents independently selected from R^{B}. In some embodiments, ring B is phenyl, optionally substituted with R^{B}. In some embodiments, ring B is phenyl.

In some embodiments, ring B is 5-6-membered heteroaryl which is optionally substituted with 1, 2, or 3 substituents independently selected from R^{B}. In some embodiments, ring B is 5-membered heteroaryl which is optionally substituted with 1, 2, or 3 substituents independently selected from R^{B}. In some embodiments, ring B is 6-membered heteroaryl which is optionally substituted with 1, 2, or 3 substituents independently selected from R^{B}. In some embodiments, ring B is 5-6-membered heteroaryl which is optionally substituted with R^{B}. In some embodiments, ring B is 5-membered heteroaryl which is optionally substituted with R^{B}. In some embodiments, ring B is 6-membered heteroaryl which is optionally substituted with R^{B}. In some embodiments, ring B is 5-6-membered heteroaryl. In some embodiments, ring B is 5-membered heteroaryl. In some embodiments, ring B is 6-membered heteroaryl. In some aspects of the foregoing embodiments, the heteroaryl is selected from thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. In other aspects of the foregoing embodiments, the heteroaryl is selected from pyridinyl (e.g., 2-pyridinyl, 3-pyridinyl, or 4-pyridinyl), pyrazinyl, pyrimidinyl, triazinyl, and pyridazinyl.

In some embodiments, ring B is selected from thienyl and pyridinyl, each of which is optionally substituted with 1, 2, or 3 independently selected R^{B}. In some embodiments, ring B is thienyl, optionally substituted with R^{B}. In some embodiments, ring B is pyridinyl, optionally substituted with R^{B}. In some embodiments, ring B is thienyl. In some embodiments, ring B is pyridinyl.

In some embodiments, the compound of Formula (I) has formula:

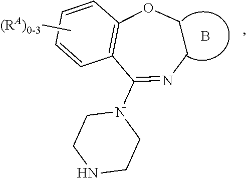

or a pharmaceutically acceptable salt thereof, wherein ring B is 5-6-membered heteroaryl as described herein.

In some embodiments, the compound of Formula (I) has formula:

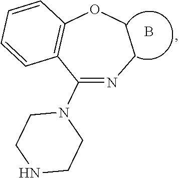

or a pharmaceutically acceptable salt thereof, wherein ring B is 5-6-membered heteroaryl as described herein.

In some embodiments, the compound of Formula (I) has formula:

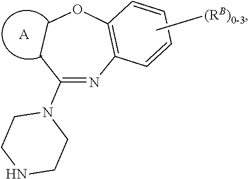

or a pharmaceutically acceptable salt thereof, wherein ring A is 5-6-membered heteroaryl as described herein.

In some embodiments, the compound of Formula (I) has formula:

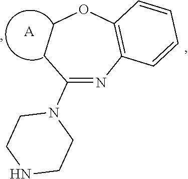

or a pharmaceutically acceptable salt thereof, wherein ring A is 5-6-membered heteroaryl as described herein.

In some embodiments, the compound of Formula (I) has formula:

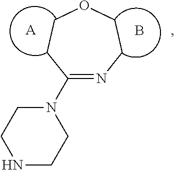

or a pharmaceutically acceptable salt thereof, wherein:
ring A is 5-6-membered heteroaryl as described herein; and
ring B is 5-6-membered heteroaryl as described herein.

In some embodiments, R^{A} is selected from OH, C₁₋₃ alkoxy, SH, NH₂, C₁₋₃ alkylamino, and di(C₁₋₃ alkyl)amino. In some embodiments, R^{A} is selected from C₁₋₃ alkyl substituted with OH, C₁₋₃ alkoxy, SH, NH₂, C₁₋₃ alkylamino, or di(C₁₋₃ alkyl)amino. In some embodiments, R^{A} is selected from OH, C₁₋₃ alkoxy, and NH₂.

In some embodiments, R^{B} is selected from OH, C₁₋₃ alkoxy, SH, NH₂, C₁₋₃ alkylamino, and di(C₁₋₃ alkyl)amino. In some embodiments, R^{B} is selected from C₁₋₃ alkyl substituted with OH, C₁₋₃ alkoxy, SH, NH₂, C₁₋₃ alkylamino, or di(C₁₋₃ alkyl)amino. In some embodiments, R^{B} is selected from OH, C₁₋₃ alkoxy, and NH₂.

In some embodiments, the compound is selected from:

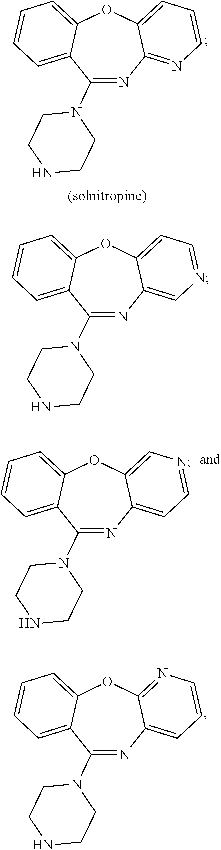

or a pharmaceutically acceptable salt thereof.

In some embodiments, the compound is selected from:

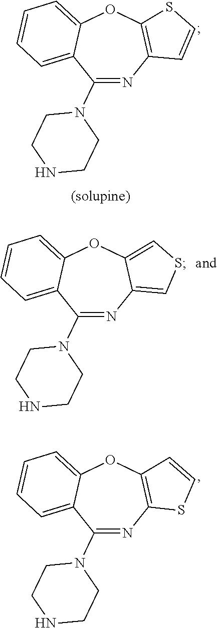 [SMILES: c1ccc2c(c1)Oc1sccc1N=C2N1CCNCC1 ; c1ccc2c(c1)Oc1cscc1N=C2N1CCNCC1 ; c1ccc2c(c1)Oc1ccsc1N=C2N1CCNCC1]

or a pharmaceutically acceptable salt thereof.

In some embodiments, the compound is selected from:

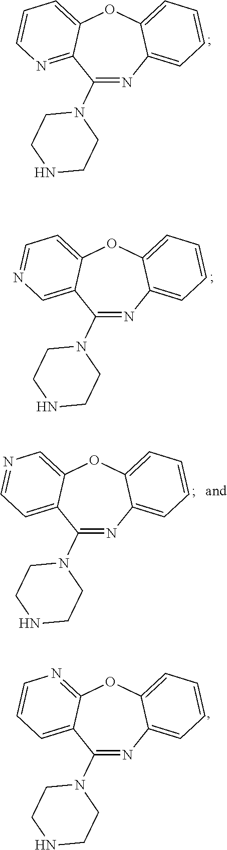 [SMILES: c1ccc2c(c1)N=C(N1CCNCC1)c1ncccc1O2 ; c1ccc2c(c1)N=C(N1CCNCC1)c1cnccc1O2 ; c1ccc2c(c1)N=C(N1CCNCC1)c1ccncc1O2 ; c1ccc2c(c1)N=C(N1CCNCC1)c1cccnc1O2]

or a pharmaceutically acceptable salt thereof.

In some embodiments, the compound is selected from:

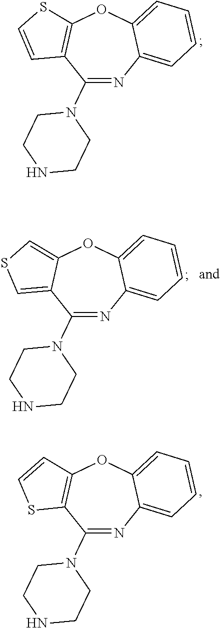 [SMILES: c1ccc2c(c1)N=C(N1CCNCC1)c1ccsc1O2 ; c1ccc2c(c1)N=C(N1CCNCC1)c1cscc1O2 ; c1ccc2c(c1)N=C(N1CCNCC1)c1sccc1O2]

or a pharmaceutically acceptable salt thereof.

In some embodiments, the compound is selected from:

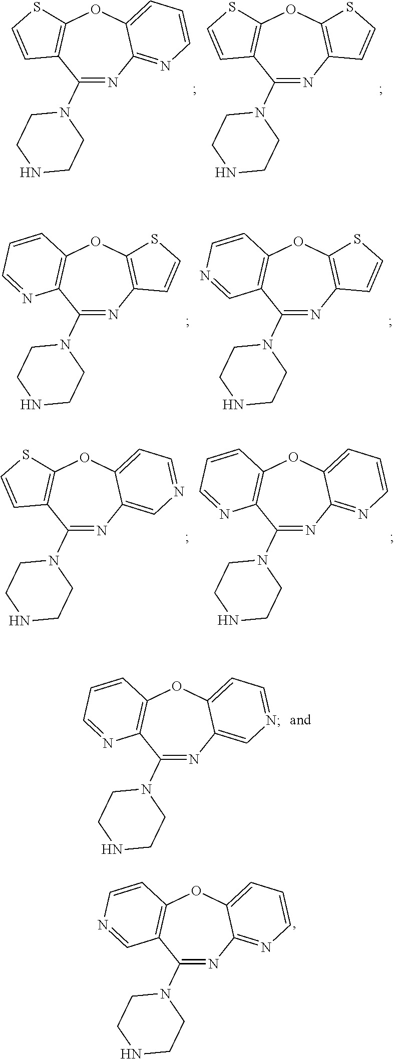

or a pharmaceutically acceptable salt thereof.

### Compound of Formula (II)

In some embodiments, the present disclosure provides a compound of Formula (II):

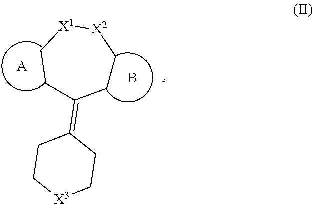

or a pharmaceutically acceptable salt thereof, wherein:
X¹ is selected from O, S, CH₂, and NR^{N};
X² is selected from O, S, CH₂, and NR^{N};
X³ is selected from O, S, and NR^{N};
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino.

In some embodiments, at least one of ring A and ring B is 5-6-membered heteroaryl.

In some embodiments, X¹ is O. In some embodiments, X¹ is S. In some embodiments, X¹ is CH₂. In some embodiments, X¹ is NR^{N}. In some embodiments, X¹ is NH.

In some embodiments, X² is O. In some embodiments, X² is S. In some embodiments, X² is CH₂. In some embodiments, X² is NR^{N}. In some embodiments, X² is NH.

In some embodiments, X¹ is CH₂ and X² is selected from O, S, and NH.

In some embodiments, X² is CH₂ and X¹ is selected from O, S, and NH.

In some embodiments, X³ is O. In some embodiments, X³ is S. In some embodiments, X³ is NR^{N}. In some embodiments, X³ is NH.

In some embodiments, the compound of Formula (II) has formula:

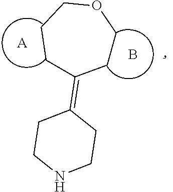

or a pharmaceutically acceptable salt thereof.

In some embodiments, the compound of Formula (II) has formula:

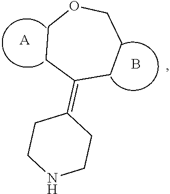

or a pharmaceutically acceptable salt thereof.

In some embodiments, ring A is phenyl, optionally substituted with 1, 2, or 3 substituents independently selected from R^{A}. In some embodiments, ring A is phenyl, optionally substituted with R^{A}. In some embodiments, ring A is phenyl.

In some embodiments, ring A is 5-6-membered heteroaryl which is optionally substituted with 1, 2, or 3 substituents independently selected from R^{A}. In some embodiments, ring A is 5-membered heteroaryl which is optionally substituted with 1, 2, or 3 substituents independently selected from R^{A}. In some embodiments, ring A is 6-membered heteroaryl which is optionally substituted with 1, 2, or 3 substituents independently selected from R^{A}. In some embodiments, ring A is 5-6-membered heteroaryl which is optionally substituted with R^{A}. In some embodiments, ring A is 5-membered heteroaryl which is optionally substituted with R^{A}. In some embodiments, ring A is 6-membered heteroaryl which is optionally substituted with R^{A}. In some embodiments, ring A is 5-6-membered heteroaryl. In some embodiments, ring A is 5-membered heteroaryl. In some embodiments, ring A is 6-membered heteroaryl. In some aspects of the foregoing embodiments, the heteroaryl is selected from thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. In other aspects of the foregoing embodiments, the heteroaryl is selected from pyridinyl (e.g., 2-pyridinyl, 3-pyridinyl, or 4-pyridinyl), pyrazinyl, pyrimidinyl, triazinyl, and pyridazinyl.

In some embodiments, ring A is selected from thienyl and pyridinyl, each of which is optionally substituted with 1, 2, or 3 independently selected R^{A}. In some embodiments, ring A is thienyl, optionally substituted with R^{A}. In some embodiments, ring A is pyridinyl, optionally substituted with R^{A}. In some embodiments, ring A is thienyl. In some embodiments, ring A is pyridinyl.

In some embodiments, ring B is phenyl, optionally substituted with 1, 2, or 3 substituents independently selected from R^{B}. In some embodiments, ring B is phenyl, optionally substituted with R^{B}. In some embodiments, ring B is phenyl.

In some embodiments, ring B is 5-6-membered heteroaryl which is optionally substituted with 1, 2, or 3 substituents independently selected from R^{B}. In some embodiments, ring B is 5-membered heteroaryl which is optionally substituted with 1, 2, or 3 substituents independently selected from R^{B}. In some embodiments, ring B is 6-membered heteroaryl which is optionally substituted with 1, 2, or 3 substituents independently selected from R^{B}. In some embodiments, ring B is 5-6-membered heteroaryl which is optionally substituted with R^{B}. In some embodiments, ring B is 5-membered heteroaryl which is optionally substituted with R^{B}. In some embodiments, ring B is 6-membered heteroaryl which is optionally substituted with R^{B}. In some embodiments, ring B is 5-6-membered heteroaryl. In some embodiments, ring B is 5-membered heteroaryl. In some embodiments, ring B is 6-membered heteroaryl. In some aspects of the foregoing embodiments, the heteroaryl is selected from thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. In other aspects of the foregoing embodiments, the heteroaryl is selected from pyridinyl (e.g., 2-pyridinyl, 3-pyridinyl, or 4-pyridinyl), pyrazinyl, pyrimidinyl, triazinyl, and pyridazinyl.

In some embodiments, ring B is selected from thienyl and pyridinyl, each of which is optionally substituted with 1, 2, or 3 independently selected R^{B}. In some embodiments, ring B is thienyl, optionally substituted with R^{B}. In some embodiments, ring B is pyridinyl, optionally substituted with R^{B}. In some embodiments, ring B is thienyl. In some embodiments, ring B is pyridinyl.

In some embodiments, the compound has formula:

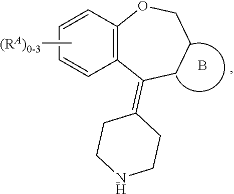

or a pharmaceutically acceptable salt thereof, wherein ring B is 5-6-membered heteroaryl as described herein.

In some embodiments, the compound has formula:

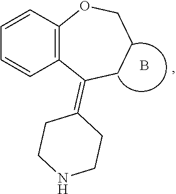

or a pharmaceutically acceptable salt thereof, wherein ring B is 5-6-membered heteroaryl as described herein.

In some embodiments, the compound has formula:

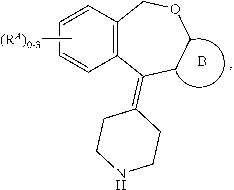

or a pharmaceutically acceptable salt thereof, wherein ring B is 5-6-membered heteroaryl as described herein.

In some embodiments, the compound has formula:

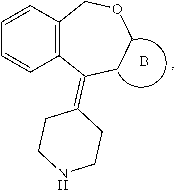

or a pharmaceutically acceptable salt thereof, wherein ring B is 5-6-membered heteroaryl as described herein.

In some embodiments, the compound has formula:

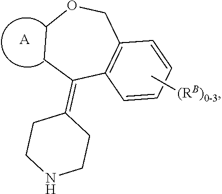

or a pharmaceutically acceptable salt thereof, wherein ring B is 5-6-membered heteroaryl as described herein.

In some embodiments, the compound of Formula (I) has formula:

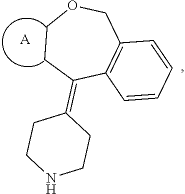

or a pharmaceutically acceptable salt thereof, wherein ring A is 5-6-membered heteroaryl as described herein.

In some embodiments, the compound of Formula (I) has formula:

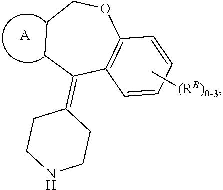

or a pharmaceutically acceptable salt thereof, wherein ring A is 5-6-membered heteroaryl as described herein.

In some embodiments, the compound of Formula (I) has formula:

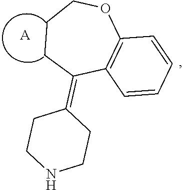

or a pharmaceutically acceptable salt thereof, wherein ring A is 5-6-membered heteroaryl as described herein.

In some embodiments, the compound of Formula (I) has formula:

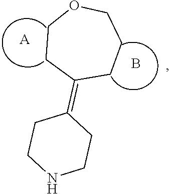

or a pharmaceutically acceptable salt thereof, wherein:
ring A is 5-6-membered heteroaryl as described herein; and
ring B is 5-6-membered heteroaryl as described herein.

In some embodiments, R^{A} is selected from OH, C₁₋₃ alkoxy, SH, NH₂, C₁₋₃ alkylamino, and di(C₁₋₃ alkyl)amino. In some embodiments, R^{A} is selected from C₁₋₃ alkyl substituted with OH, C₁₋₃ alkoxy, SH, NH₂, C₁₋₃ alkylamino, or di(C₁₋₃ alkyl)amino. In some embodiments, R^{A} is selected from OH, C₁₋₃ alkoxy, and NH₂.

In some embodiments, R^{B} is selected from OH, C₁₋₃ alkoxy, SH, NH₂, C₁₋₃ alkylamino, and di(C₁₋₃ alkyl)amino. In some embodiments, R^{B} is selected from C₁₋₃ alkyl substituted with OH, C₁₋₃ alkoxy, SH, NH₂, C₁₋₃ alkylamino, or di(C₁₋₃ alkyl)amino. In some embodiments, R^{B} is selected from OH, C₁₋₃ alkoxy, and NH₂.

In some embodiments, the compound of Formula (II) is selected from:

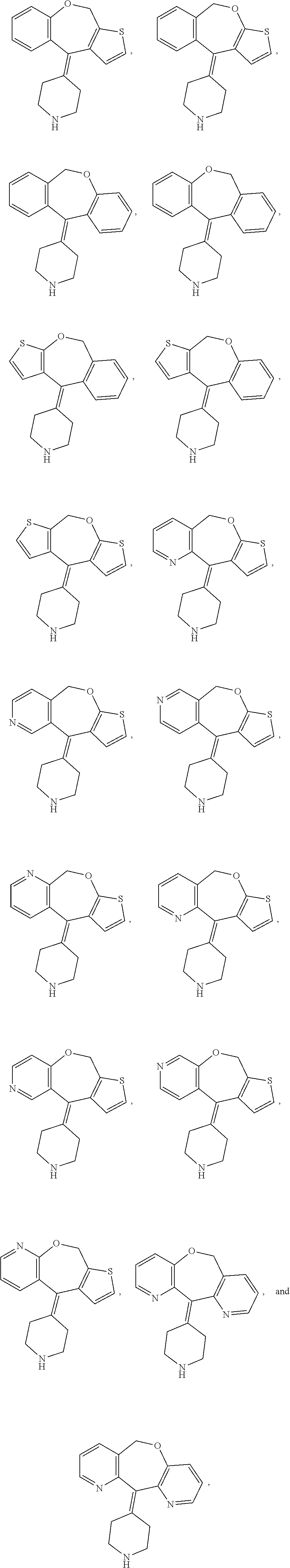

or a pharmaceutically acceptable salt thereof.

### Pharmaceutically Acceptable Salts

In some embodiments, a salt of a compound of this disclosure is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of this disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of this disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. In some embodiments, the compounds described herein, or pharmaceutically acceptable salts thereof, are substantially isolated.

### Pharmaceutical Compositions

The present application also provides pharmaceutical compositions comprising an effective amount of a compound of the present disclosure, or a pharmaceutically acceptable salt thereof, and a pharmaceutically acceptable carrier. The pharmaceutical composition may also comprise at least one of any one of the additional therapeutic agents described. In certain embodiments, the application also provides pharmaceutical compositions and dosage forms comprising any one the additional therapeutic agents described herein (e.g., in a kit). The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, intraocular, intracameral, periocular, intravitreal, subconjunctival, subtenon, retrobulbar, intrascleral, suprachoroidal, subretinal nasal, nasogastric, oral, parenteral, percutaneous, peridural, rectal, respiratory (inhalation), subcutaneous, sublingual, submucosal, topical, transdermal, transmucosal, transtracheal, ureteral, urethral, vaginal, and ocular.

Compositions and formulations described herein may conveniently be presented in a unit dosage form, e.g., tablets, capsules (e.g., hard or soft gelatin capsules), sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

In some embodiments, the present disclosure provides a pharmaceutical formulation of a compound described herein that is suitable for ocular (topical) administration (e.g., administration by an ocular route). Suitable examples of such formulations include eye-drops, eye ointment, and eye emulsion. The formulation contains additional ingredients that allow the compound to permeate into main ocular circulatory system and cross the ocular barrier. In some embodiments, the compound can be coated on a contact lens. In some embodiments, the compound can be administered by a local injection into or near the cornea, choroid, retina, vitreous, uvea, orbit, eyelid, conjunctiva, or iris. Suitable examples of intraocular routes include: intravitreal, intraocular, periocular, intracameral, retrobulbar, subconjunctival, subtenon, intracorneal, intrastromal, trans-scleral, subretinal, and suprachoroidal route. Any of the formulations described herein can be administered by any of these routes. The compound can be coated on any implant, stent or drainage device placed in or around the eye or orbit. The compound can be coated on a contact lens or scleral lens or a punctal plug. The compound can also be made into a sustained release delivery device on any of the aforementioned routes or devices. The compound can be topical eye drops or gels or coated on cotton tips or Weck-cells or similar applicators on the surface of the eye. The compound can be delivered by nanoparticle delivery devices or ultrasound or electrical stimulation. The compound could be light activated or activated or delivered by any known delivery route currently available.

### Dosages and Regimens

In the pharmaceutical compositions of the present application, a therapeutic compound is present in an effective amount (e.g., a therapeutically effective amount).

Effective doses may vary, depending on the diseases treated, the severity of the disease, the route of administration, the sex, weight, height, age and general health condition of the subject, excipient usage, the possibility of co-usage with other therapeutic treatments such as use of other agents and the judgment of the treating physician.

In some embodiments, an effective amount of a therapeutic compound can range, for example, from about 0.00005 mg/kg to about 0.0001 mg/kg, from about 0.0001 mg/kg to about 0.0005 mg/kg, or from about 0.0001 mg/kg to about 0.0005 mg/kg. In some aspects of these embodiments, the effective amount is administered by an intravitreal injection. In some embodiments, an effective amount is from about 0.00001 mg/kg to about 5 mg/kg; from about 0.0001 mg/kg to about 1 mg/kg; from about 0.0001 mg/kg to about 0.5 mg/kg; from about 0.00001 mg/kg to about 0.01 mg/kg; or from about 0.0001 mg/kg to about 0.001 mg/kg. In some embodiments, an effective amount of a therapeutic compound is about 0.00005 mg/kg, about 0.0001 mg/kg, about 0.0005 mg/kg, about 0.001 mg/kg, or about 0.1 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month). The compounds and compositions described herein can be administered to the subject in any order. A first therapeutic agent, such as a compound of the present disclosure, can be administered prior to or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before or after), or concomitantly with the administration of a second therapeutic agent, such as an anti-fibrotic agent described herein, to a subject in need of treatment. Thus, the compound of the present disclosure, or a composition containing the compound, can be administered separately, sequentially or simultaneously with the second therapeutic agent, such as an anti-fibrotic agent described herein. When the compound of the present disclosure, or a pharmaceutically acceptable salt thereof, and a second or third therapeutic agent are administered to the subject simultaneously, the therapeutic agents may be administered in a single dosage form (e.g., tablet, capsule, or a solution for injection or infusion).

In some embodiments, the second (additional) therapeutic agent is a drug that is useful in treating or preventing an ocular fibrotic pathology. In some embodiments, the second therapeutic is selected from 5-fluorouracil (5-FU), daunorubicin, taxol, colchicine, retinoic acid, ribozymes, vincristine, cisplatin, adriamycin, mitomycin, dactomycin, and methotrexate, or a pharmaceutically acceptable salt thereof. In some embodiments, the additional therapeutic agent is dopamine, or a pharmaceutically acceptable salt thereof. In some embodiments, the additional therapeutic agent is a dopamine receptor antagonist. In some embodiments, the second (additional) therapeutic agent is an anti-inflammatory drug. Suitable examples of such drugs include NSAIDs such as celecoxib, rofecoxib, ibuprofen, naproxen, aspirin, diclofenac, sulindac, oxaprozin, piroxicam, indomethacin, meloxicam, fenoprofen, diflunisal, methotrexate, BAY 11-7082, or a pharmaceutically acceptable salt thereof. Suitable examples of steroid anti-inflammatory agents include cortisol, corticosterone, hydrocortisone, aldosterone, deoxycorticosterone, triamcinolone, bardoxolone, bardoxolone methyl, triamcinolone, cortisone, prednisone, and methylprednisolone, or a pharmaceutically acceptable salt thereof. In some embodiments, the additional therapeutic agent (e.g., the second or third therapeutic agent) can be an agent used to treat the initial cause of the ocular fibrotic pathology. For example, if the ocular fibrosis is due to or related to angiogenesis or neovascularization or ocular inflammation, the compound of this disclosure can be administered before, at the same time, or after the anti-angiogenic or the anti-inflammatory agent.

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. Kits can also include various items, compounds, and devices that may be needed for administering the drug. Examples of such items include anesthestic, antiseptic, cotton tipped applicators or swabs, gauze, balanced salt solutions, syringes, eyelid speculum and the like.

### Definitions

As used herein, the term “about” means “approximately” (e.g., plus or minus approximately 10% of the indicated value).

As used herein, the term “compound” as used herein is meant to include all stereoisomers, geometric isomers, tautomers, and isotopes of the structures named or depicted. Compounds herein identified by name or structure as one particular tautomeric form are intended to include other tautomeric forms unless otherwise specified.

The terms “pharmaceutical” and “pharmaceutically acceptable” are employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal. In some embodiments, the cell is a mesenchymal cell. In some embodiments, the cell is a fibroblast (e.g., cardiac, dermal or lung fibroblast). In some embodiments, the cell is a hepatic stellate cell.

As used herein, the term “contacting” refers to the bringing together of indicated moieties or items in an in vitro system, an ex vivo system, or an in vivo system. For example, “contacting” a cell with a compound provided herein includes the act of administering that compound to a mammal (e.g., a human) containing that cell as well as, for example, introducing that compound into a cell culture containing that cell.

As used herein, the term “mammal” includes, without limitation, mice, rats, other rodents, rabbits, dogs, cats, swine, cattle, sheep, horses, elephants, deer, non-human primates (e.g., monkeys and apes), house pets, and humans.

As used herein, the phrase “effective amount” or “therapeutically effective amount” refers to the amount of active compound or pharmaceutical agent that elicits the biological or medicinal response in a tissue, system, mammal, or human that is being sought by a researcher, veterinarian, medical doctor, or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, without limitation, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms that may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Suitable examples of alkylamino groups include N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di Cₙ₋ₘ alkylamino” refers to a group of formula —N(alkyl)₂, wherein each alkyl group independently has n to m carbon atoms. In some embodiments, each alkyl group has 1 to 6, 1 to 4, or 1 to 3 carbon atoms. Suitable examples of dialkylamino groups include N,N-methylehtylamino, N,N-diethylamino, N,N-propylethylamino, N,N-butylisopropylamino, and the like.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “HO—C₁₋₃ alkyl” refers to a group of formula —(C₁₋₃ alkylene)-OH.

As used herein, the term “NH₂—C₁₋₃ alkyl” refers to a group of formula —(C₁₋₃ alkylene)-NH₂.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur, and oxygen. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from nitrogen, sulfur, and oxygen. In some embodiments, the heteroaryl is a 5-6 monocyclic heteroaryl having 1 or 2 heteroatom ring members independently selected from nitrogen, sulfur, and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls include, without limitation, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls include, without limitation, pyridyl, pyrazinyl, pyrimidinyl, triazinyl, and pyridazinyl.

### EXAMPLES

### Materials and Methods

### Cell Culture

The human RPE cell line ARPE-19 was purchased from ATCC and cultured in Dulbecco's Modified Eagle Medium/Nutrient Mixture F-12 (DMEM/F-12) supplemented with 2.50 mM L-Glutamine, 15 mM HEPES Buffer, 10% fetal bovine serum (FBS), and 1% antibiotic-antimycotic (Gibco), unless otherwise noted. Cells were maintained in a humidified 37° C., 5% CO₂ incubator. All experiments were performed with cells at passage 3-6.

### Chemicals and Reagents

Dimethyl sulfoxide (DMSO) was purchased from Fisher Scientific and used to solubilize loxapine and pramipexole. 2-mercaptoethanol was purchased from Bio-Rad Laboratories and added to RLT buffer prior to RNA isolation, as per Qiagen instructions. Pramipexole (hydrochloride) and loxapine, were purchased from Cayman Chemical Company. TGFβ was purchased from InvivoGen. Loxapine has been shown to be a very stable molecule in vitro and in vivo.

RNA Isolation qPCR Analysis

ARPE-19 cells were plated into 12 well plates (100,000 cells/well) and allowed to attach. Media was removed and replaced with the media specified in the “Cell Culture” section above, minus FBS. Cells were treated for 24 hours±TGFβ (10 ng/mL), 1 μM pramipexole, and 10 μM loxapine. RNA was isolated using the RNeasy Plus Mini Kit (Qiagen) and the manufacturer's protocol. Isolated RNA was converted to cDNA using the SuperScript VILO cDNA Synthesis Kit (Invitrogen) and the PTC-200 Peltier Thermal Cycler (MJ Research). Quantitative PCR (qPCR) was performed using FastStart Essential DNA Green Master (Roche) and LightCycler 96 (Roche). Data are expressed as a fold change by ΔΔCt relative to GAPDH. qPCR primers (IDT) are shown in Table 1.

TABLE 1
Primers used in qPCR Analysis

| | Sequence |
|---|---|
| GAPDH | Forward: GGAAGGGCTCATGACCACAG |
| | Reverse: ACAGTCTTCTGGGTGGCAGTG |
| COL1A1 | Forward: AAGGGACACAGAGGTTTCAGT GG |
| | Reverse: CAGCACCAGTAGCACCATCATTTC |
| ACTA2 | Forward: GTGAAGAAGAGGACAGCACTG |
| | Reverse: CCCATTCCCACCATC ACC |
| CTGF | Forward: GTCCAGCACGAGGCTCA |
| | Reverse: TCGCCTTCGTGGTCCTC |
| FN1 | Forward: TGTCAGTCAAAGCAAGCCCG |
| | Reverse: TTAGGACGCTCATAAGTGTCACCC |

### Proliferation and Live Dead Assays

ARPE-19 cells were plated into 96-well plates (1,000 cells/well) and allowed to attach. Media was removed and replaced with the media specified in the “Cell Culture” section above, with reduced FBS. Cells were treated for four days±2% FBS, 10 μM loxapine. After four days, CellTiter-Fluor™ (Promega) was added to each well according to the manufacturer and measured on a Flexstation 3 (Molecular Devices) plate reader for the proliferation assay. For the LIVE/DEAD™ assay, each component of the kit (Thermo Fisher Scientific) was added, then cells were incubated for 30 minutes. Fluorescent images were then taken using a Cytation 5 imaging reader (BioTek) and quantified for cytotoxicity/viability.

### Fibronectin Deposition

ARPE-19 cells were plated into 96-well plates (10,000 cells/well) and allowed to attach. Media was removed and replaced with the media specified in the “Cell Culture” section above, without FBS. Cells were treated for four days±TGFβ (10 ng/mL) and 10 μM loxapine. Cells were fixed with 10% neutral buffered formalin (Sigma-Aldrich) for 15 minutes. Cells were permeabilized with 0.25% Triton X-100 (Sigma-Aldrich) and blocked with 1% BSA for 1 hour. Cells were incubated with mouse monoclonal IgG₃ primary antibodies against fibronectin (Santa Cruz Biotechnology), which were diluted 1:400 in 0.25% Triton X-100 (Sigma-Aldrich) with 1% BSA. Cells were incubated with primary antibodies either in 4° C. overnight or at room temperature for 2 hours. Cells were washed with PBS (Gibco), then incubated for 1 hour with Alexa Fluor® 488 donkey anti-mouse IgG (H+L) secondary antibodies (Life Technologies) and DAPI (Thermo Fisher Scientific), which were each diluted 1:1000 in 0.25% Triton X-100 (Sigma-Aldrich) with 1% BSA. Cells were washed again with PBS (Gibco). Immunofluorescence was measured using a Cytation 5 imaging reader (BioTek).

### Wound Healing Assay

ARPE-19 cells were plated confluent into a 12-well plate (300,000 cells/well) and allowed to attach. Cells were exposed to a single scratch made with a p200 pipette tip. Media was removed and replaced with the media specified in the “Cell Culture” section above, without FBS. Cells were treated±TGFβ (10 ng/mL) and 10 μM loxapine. Cells were imaged after treatment for 0, 24, 48, and 72 hours. Cells were imaged with an inverted phase-contract microscope and wound area was measured using ImageJ software as previously described.

### Dopamine ELISA Assay

ARPE-19 cells were plated into a 12-well plate (300,000 cells/well) and allowed to attach. Media was removed and replaced with the media specified in the “Cell Culture” section above, without FBS. Cells were treated±TGFβ (10 ng/mL). Media was collected from cultured cells after treatment for 24 hours. Dopamine levels in the media were measured using the Mouse Dopamine ELISA kit (MyBioSource) and the manufacturer's protocol. Dopamine was quantified using the FlexStation 3 plate reader (Molecular Devices).

### Statistics

In experiments comparing groups of three or more, groups were compared by one-way analysis of variance with Tukey's post-hoc comparison after confirming that data displayed a normal distribution. In experiments comparing two groups, data were compared using t-test with Welch's correction. Results are expressed throughout as the mean±standard error of the mean (SEM) with each individual datapoints shown. Statistical tests were carried out using GraphPad Prism 9 with statistical significance defined in each figure legend.

### Example 1

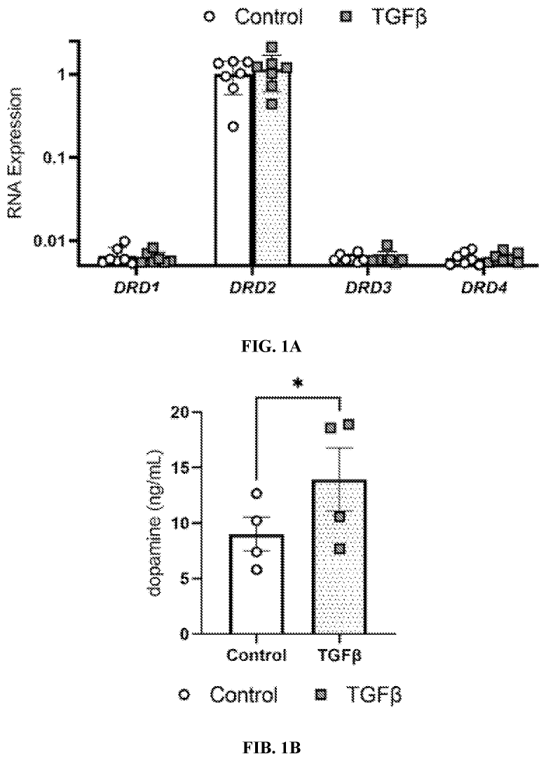

Results. To determine D2 dopamine receptor presence in cultured RPE cells, transcript levels were measured by qPCR, ±TGFβ to compare expression in unstimulated, and profibrotic conditions. DRD2 is dominantly expressed in these cells (FIG. 1A) compared to DRD1,3,4. These results show that D2 dopamine receptors are present in RPE cells and have potential to regulate their fibrotic activation. To evaluate dopamine production in RPE cells, ELISA assay was performed on the conditioned media collected from ARPE-19 cells treated±TGFβ for 24 hours. Data demonstrate that ARPE-19 cells produced dopamine (FIG. 1B), as was previously shown by HPLC analysis. Moreover, they exhibited enhanced dopamine production in the presence of TGFβ (FIG. 1), showing that dopamine receptor signaling regulates the fibrotic activity of RPE cells.

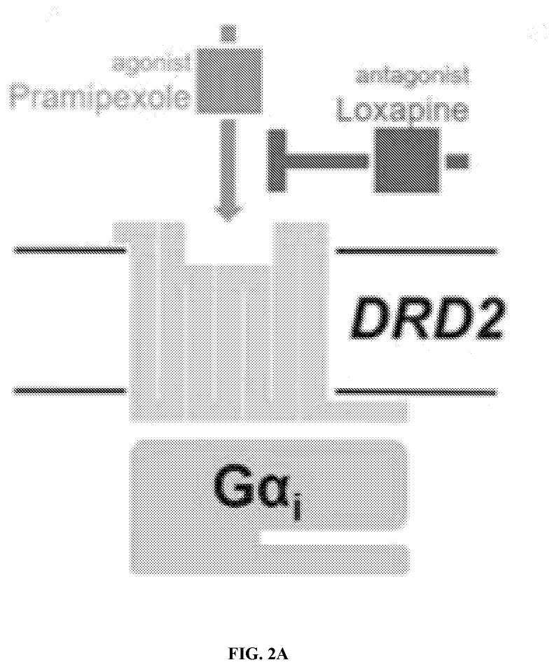

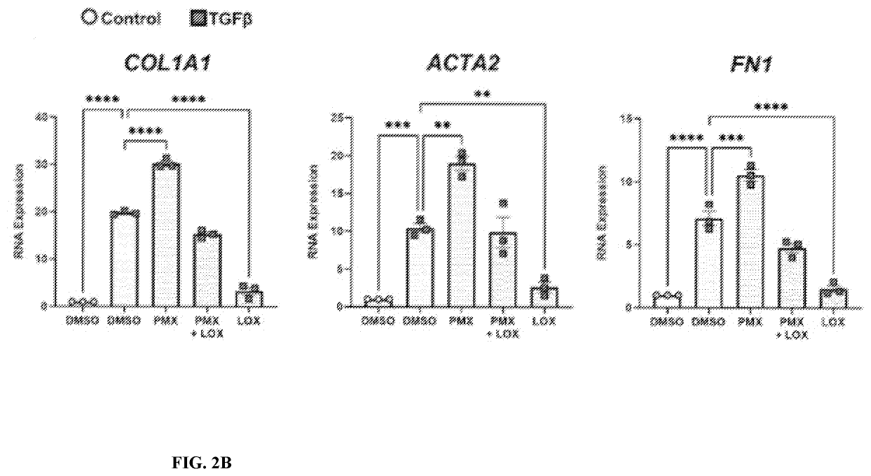

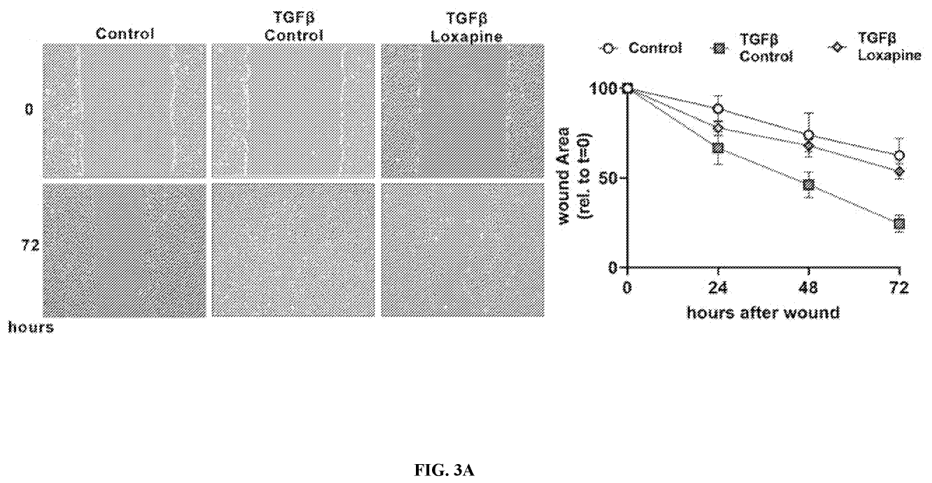

After identifying the preferential expression of DRD2 in ARPE-19 cells, the regulatory effects of these receptors on fibrotic activation were elucidated. As to D2 dopamine receptor (FIG. 2A-B), RNA isolation and qPCR were used to measure the transcript levels of fibrotic markers in ARPE-19 cells treated±TGFβ, pramipexole (D2 agonist), and loxapine (D2 antagonist). TGFβ treatment alone dramatically enhanced expression of COL1A1 (type I collagen), ACTA2 (α-smooth muscle actin), and FN1 (fibronectin), consistent with previous findings identifying TGFβ being a major contributor to epithelial-mesenchymal transition and subsequent fibrosis associated with PVR. The D2 receptor agonist combined with TGFβ even further elevated expression of these genes. Conversely, ARPE-19 cells treated with TGFβ and the D2 receptor antagonist exhibited a significant decrease in the expression of COL1A1, ACTA2, and FN1 compared to those treated with TGFβ only (FIG. 2B). Since TGFβ was shown to induce a significant upregulation increase in the expression of COL1A1, ACTA2, and FN1 in our cells (FIG. 2B), these findings suggest that when stimulated into a fibrotic state by TGFβ, D2 dopamine receptor agonism accentuates fibrotic activity in RPE cells while D2 dopamine receptor antagonism inhibits this response. In experiments combining pramipexole and loxapine along with TGFβ negates the activity of the molecules alone, confirming their activity is being driven by their interaction with the D2 receptor (FIG. 2B). The robust effect of loxapine also suggests the dopamine being produced by these cells in culture, autocrine signals through the D2 receptor.

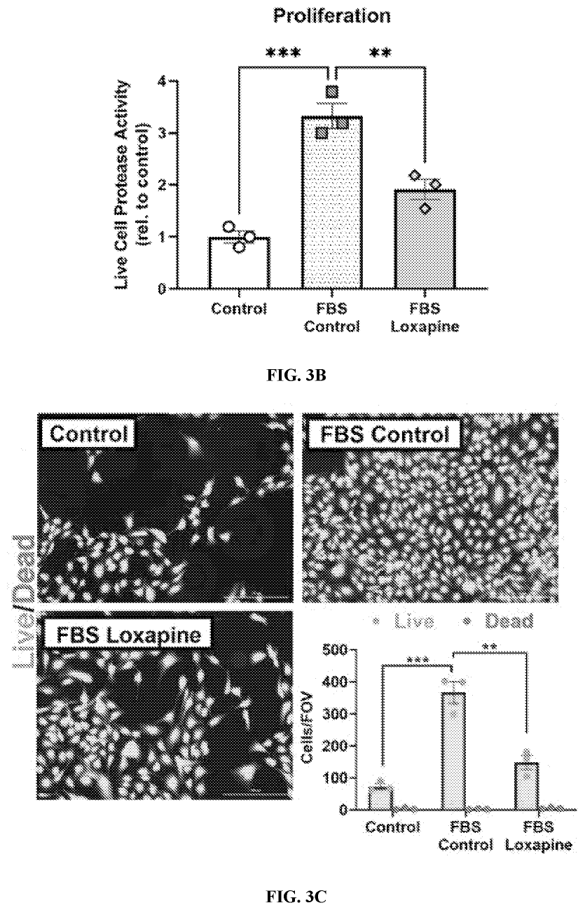

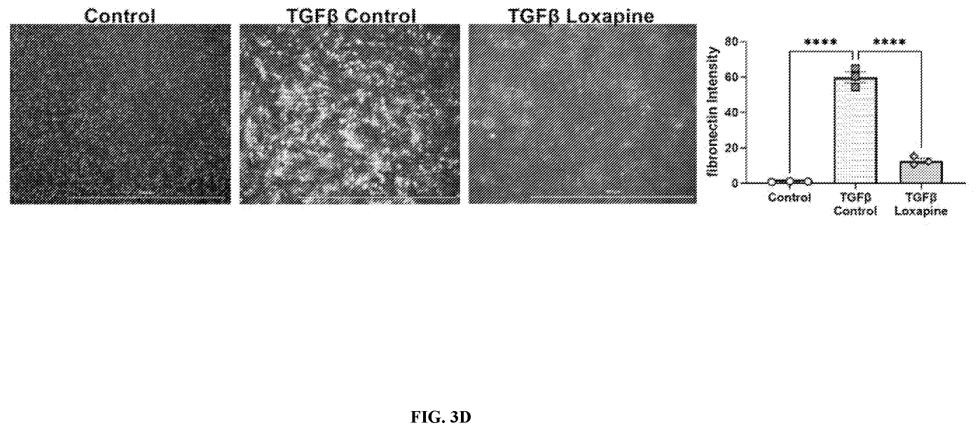

Based on the results, a potential route to inhibit TGFβ mediated fibrotic responses in cultured RPE cells is antagonism of the D2 receptor (loxapine). To confirm this robust antifibrotic strategy an in vitro assay was carried out to capture the pathogenic phenotypes of RPE cells in PVR: enhanced migration, aberrant proliferation, and excess ECM deposition. 2-dimensional cell migration was measured using a scratch-wound assay. ARPE-19 stimulated with TGFβ, migrated into the open “wound” area at a faster rate than unstimulated cells, treating the cells with loxapine halted the enhanced migration (FIG. 3A). Loxapine also blocks serum stimulated cellular proliferation which was quantified using two methods, first measuring live-cell protease activity, and second using a LIVE/DEAD assay. In both analysis fetal bovine serum enhanced proliferation 3-4-fold and loxapine effectively inhibited this cellular expansion over the 4 day exposure. To confirm this effect is not simply a cytotoxic response to the compound, the number of dead cells were measured which stain red after ethidium homodimer-1 exposure compared to the number of live cells which stain green after calcein-AM exposure. Loxapine blocks proliferation of RPE cells without promoting a cytotoxic response (FIG. 3C). To measure ECM deposition, ARPE-19 cells were plated at confluence for 4 days in the presence of TGFβ±loxapine and immunostained for fibronectin, a major extracellular matrix protein abundant in fibrotic PVR membranes. A robust enhancement of fibronectin deposition was observed in response to TGFβ, consistent with the increase in FN1 transcript levels shown in FIG. 2. Loxapine dramatically repressed the deposition of fibronectin in this assay (FIG. 3D).

### Discussion

Taken together, the results highlight D2 as therapeutic target for the treatment of retinal fibrotic diseases involving RPE cells, including proliferative vitreoretinopathy. DRD2 is preferentially expressed in RPE cells. Moreover, DRD2 activation enhances profibrotic activity. These results are consistent with previous studies, which have detected DRD2 expression in RPE cells. Additionally, DRD2 is involved in promoting fibrosis. For instance, one group found that DRD2 inhibition attenuated fibrosis in diabetic hepatic stellate cells. Another study found that DRD2 antagonism blocked fibrosis in a minipig non-alcoholic steatohepatitis model.

The results from this study, which implicate dopamine receptor activation as a regulator of retinal fibrosis, are also consistent with findings indicating enhanced levels of 3,4-dihydroxyphenylacetic acid, a surrogate index of retinal dopamine, in patients with rhegmatogenous retinal detachment (RRD), dramatically higher than patients with macular pucker and vitreous hemorrhage. Because PVR is preceded by RRD, these findings suggest that upregulated dopamine levels during RRD may preferentially activate profibrotic receptors such as DRD2, contributing to the pathogenesis of PVR.

The results highlight the promising therapeutic potential of DRD2 antagonists. Loxapine, for example, is a “typical antipsychotic” that has been used clinically for over 40 years and is delivered by intramuscular injection, oral capsule, oral concentration, or inhaled powder suggesting it is highly amenable to novel formulations or deliveries. Although originally characterized as a selective D2 antagonist recent evidence suggests it also mediates it antipsychotic effects partially through serotonin receptor antagonism. In the results, combining D2 agonist with loxapine blocked the antifibrotic effects (FIG. 2B), suggesting its activity is primarily driven through antagonism of the D2 receptors in RPE cells.

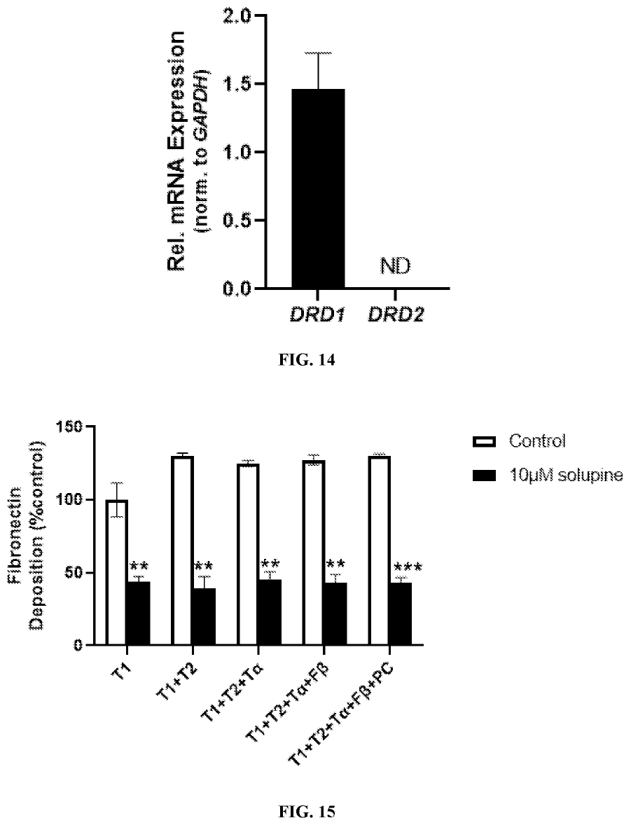

FIG. 14 shows mRNA expression of DRD1 and DRD2 in corneal epithelia. RNA isolated from Surgical waste samples of patient corneal epithelial were analyzed for transcript expression of dopamine receptors DRD1 and DRD2. N=3 unique patients. Results: DRD2 is not expressed in corneal epithelia.

### Example 2

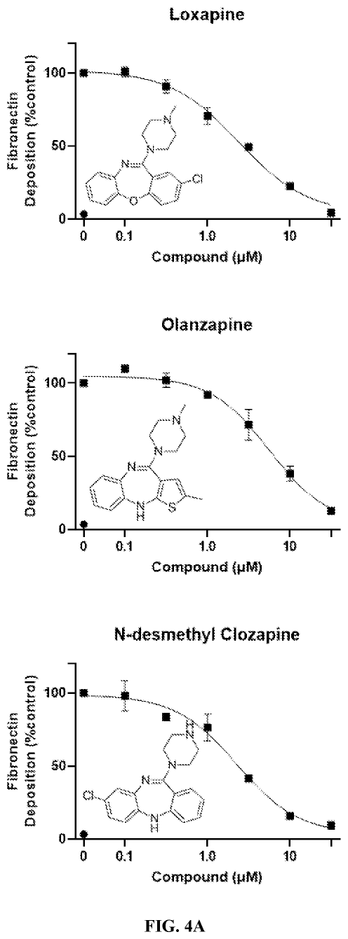

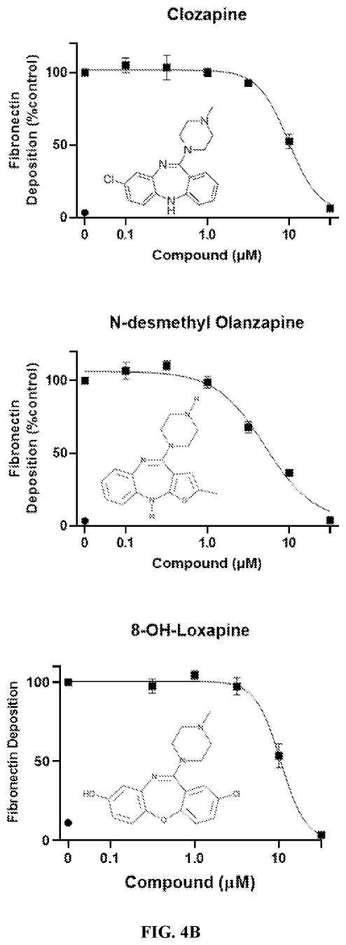

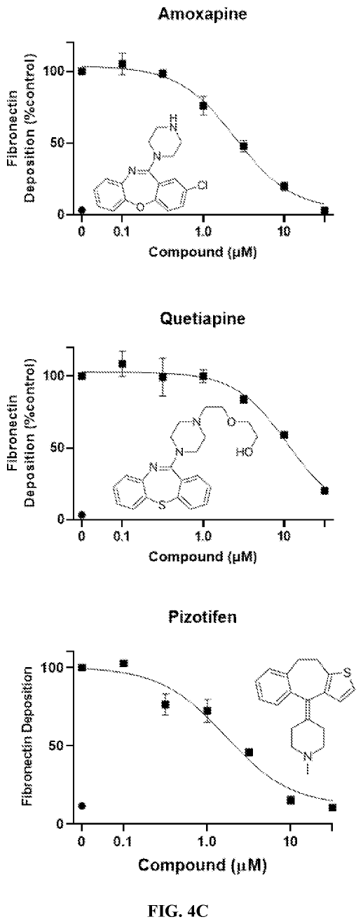

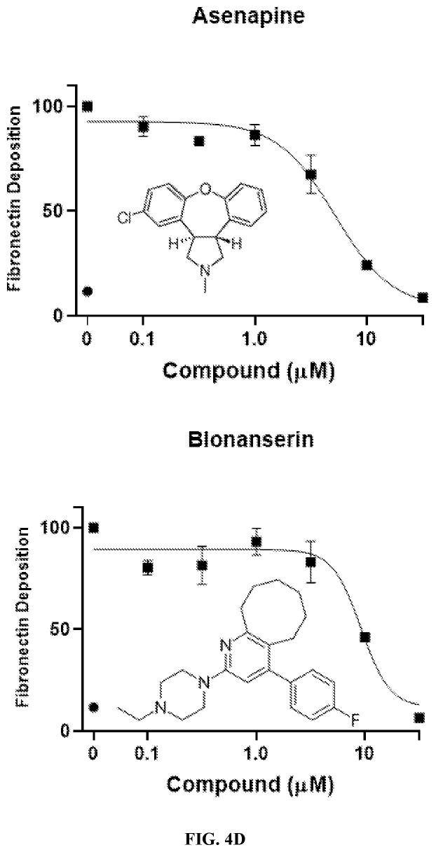

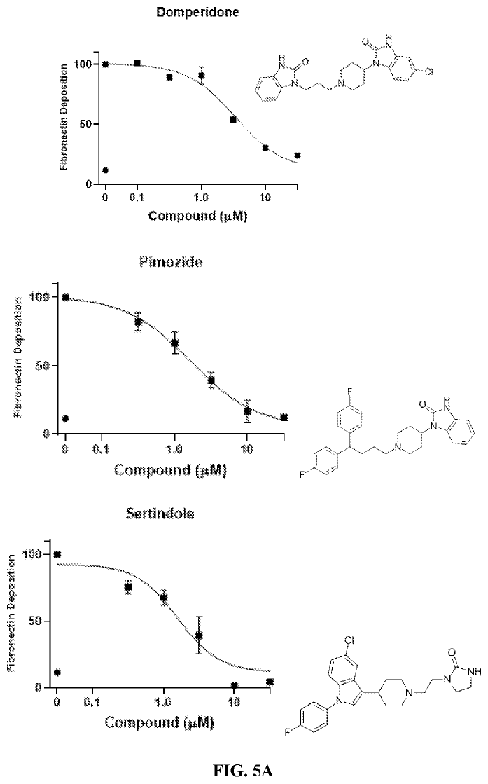

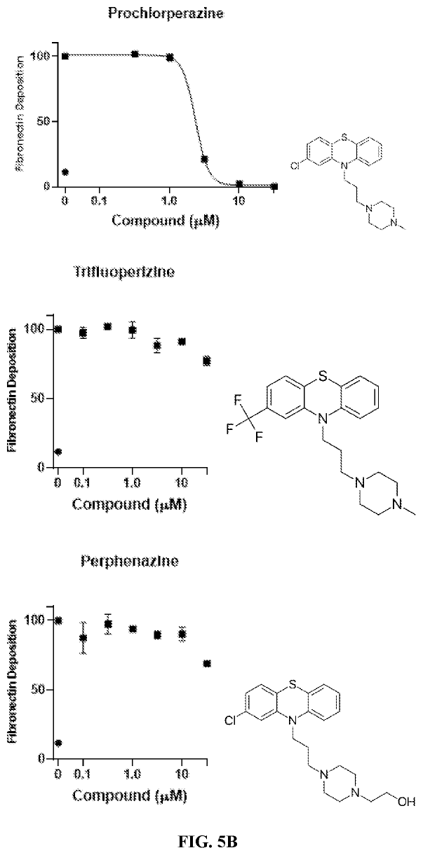

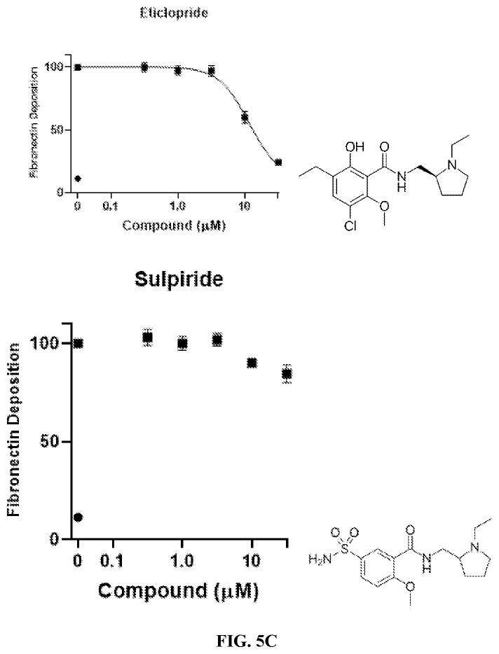

The compounds of this Example were tested using the fibronectin deposition assay described in Example 1. In brief, ARPE-19 cells were stimulated with TGFβ+/−the indicated concentration of each compound. Incubated 96 hours Fixed and immunostained for fibronectin. Results of the assay for compounds loxapine, clozapine, amoxapine, olanzapine, N-desmethyl olanzapine, quetiapine, N-desmethyl clozapine, 8-OH-loxapine, pizotifen, asenapine, blonanserin, domperidone, pimozide, sertindole, eticlopride, prochlorperazine, trifluoperizine, perphenazine, remoxipride, amisulpride, raclopride, sulpiride, methotrexate, spiperone, fluspirilene, penfluridol, droperidol, timiperone, benperidol, aripiprazole, and lurasidone are shown in FIGS. 4A-6B and 9A-9C. IC₅₀ values are presented in Table 1.

TABLE 1

| Compound | IC₅₀, μM |
|---|---|
| loxapine | + |
| clozapine | ++ |
| amoxapine | + |
| olanzapine | ++ |
| N-desmethyl olanzapine | + |
| quetiapine | +++ |
| N-desmethyl clozapine | + |
| 8-OH-loxapine | +++ |
| pizotifen | + |
| asenapine | ++ |
| blonanserin | ++ |
| domperidone | + |
| pimozide | + |
| sertindole | + |
| eticlopride | +++ |
| prochlorperazine | + |
| trifluoperizine | +++ |
| perphenazine | +++ |
| remoxipride | +++ |
| amisulpride | +++ |
| raclopride | +++ |
| sulpiride | +++ |
| solnitropine | + |
| solupine | + |
| methotrexate | +++ |
| spiperone | ++ |
| fluspirilene | + |
| penfluridol | + |
| droperidol | ++ |
| timiperone | + |
| benperidol | + |
| lurasidone | + |

“+” ≤5 μM
“++” 5 to 10 μM
“+++” 10 2 μM

### Example 3

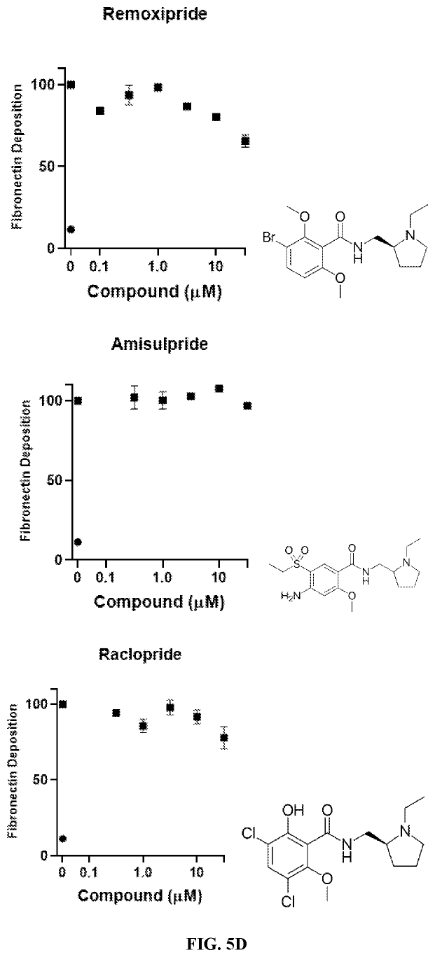

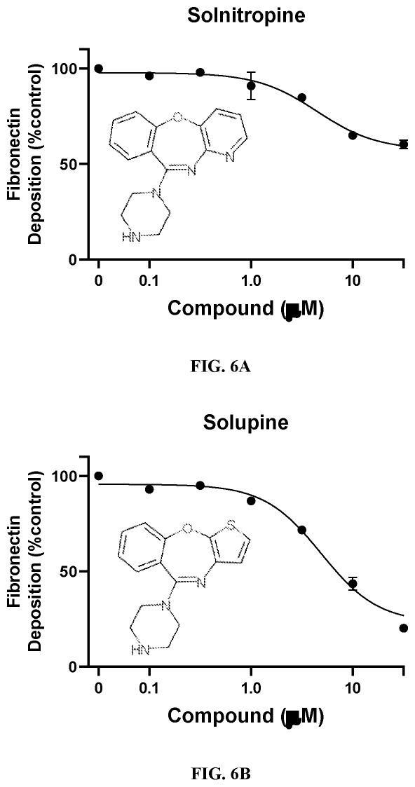

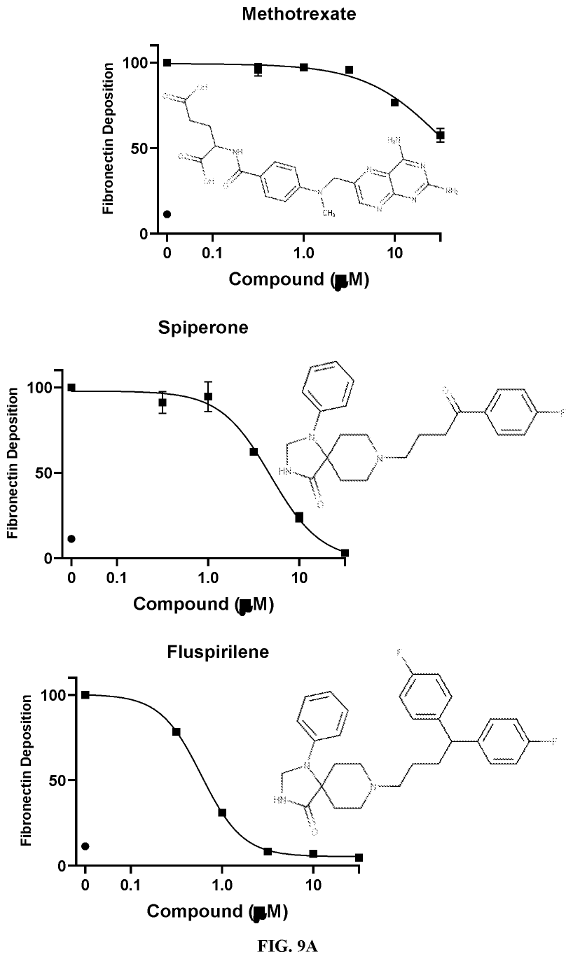

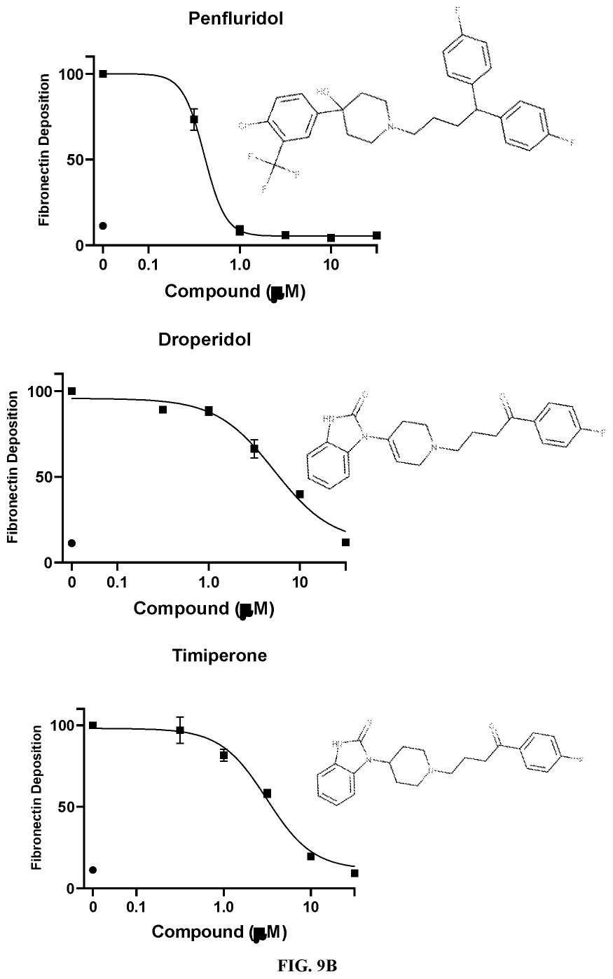

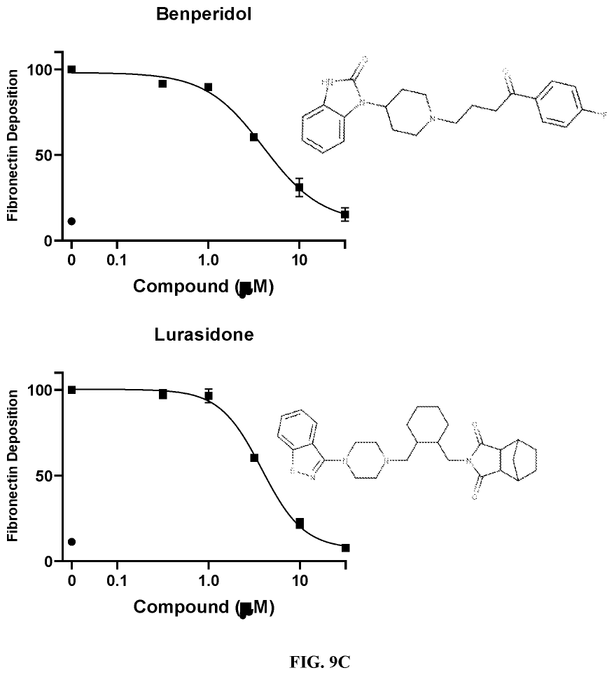

The antifibrotic activity of novel D2 dopamine receptor antagonists solnitropine and solupine were evaluated using the fibronectin deposition assay described in Example 1 (FIGS. 6A and 6B). The antifibrotic activity of D2 dopamine receptor antagonists methotrexate, spiperone, fluspirilene, penfluridol, droperidol, timiperone, benperidol, and lurasidone were evaluated using the fibronectin deposition assay described in Example 1 (FIG. 9A9C). In brief, ARPE-19 cells were stimulated with TGFβ+/−the indicated concentration of each compound. Incubated 96 hours, fixed and immunostained for fibronectin. Both compounds demonstrated IC₅₀ values ≤5 μM.

### Example 4

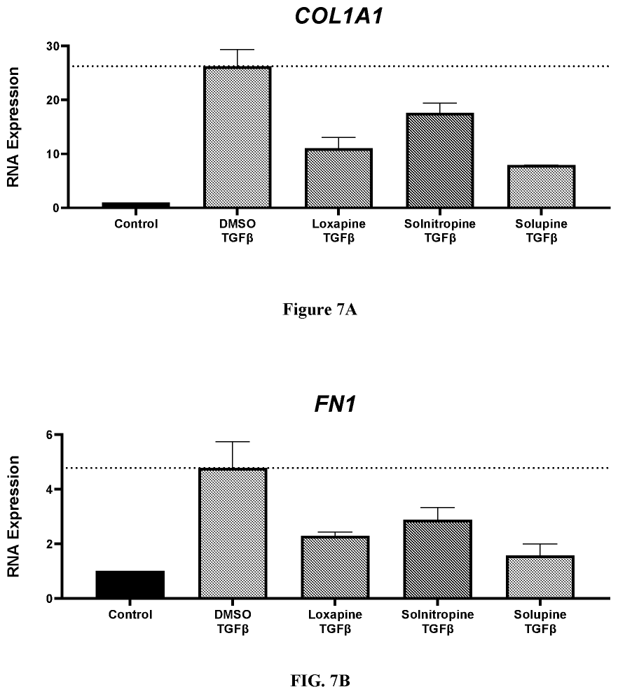

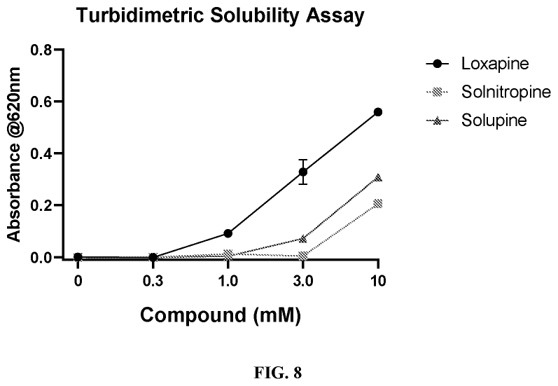

The effect of solnitropine, solupine, and loxapine on the expression of profibrotic genes COL1A1 and FN1 were evaluated as described in Example 1 (FIGS. 7A and 7B). In brief, ARPE-19 cells were treated±TGFβ in the presence of either loxapine, solnitropine, or solupine. Gene expression was measured by qPCR on RNA isolated from the treated cells 24 h after treatment. Treatment with TGFβ alone resulted in elevated levels of both COL1A1 and FN1, however co-treatment with any of the three D2 dopamine receptor antagonists significantly reduced the expression of both genes.

### Example 5

Comparison of the aqueous solubility of loxapine, solnitropine, and solupine. Compounds were incubated for 20 minutes in phosphate buffered saline (pH=7.4) at the indicated concentration. The absorbance was measured at 620 nm using a spectrophotometer. As less soluble compounds form precipitates, they increase the turbidity of the solution and increase absorption at 620 nm as described in (Kearns and Di, JALA, 2005; 10 (2): 114-123). Solnitropine and solupine are ˜3-10 fold more soluble in aqueous solution than loxapine.

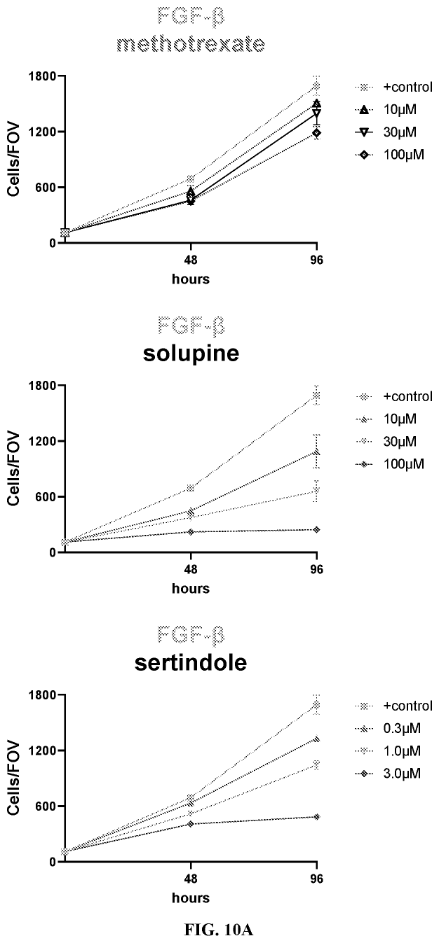

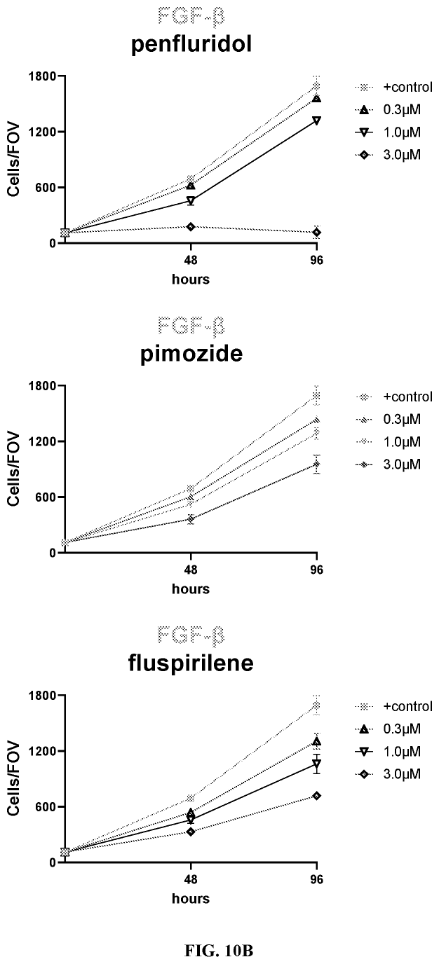

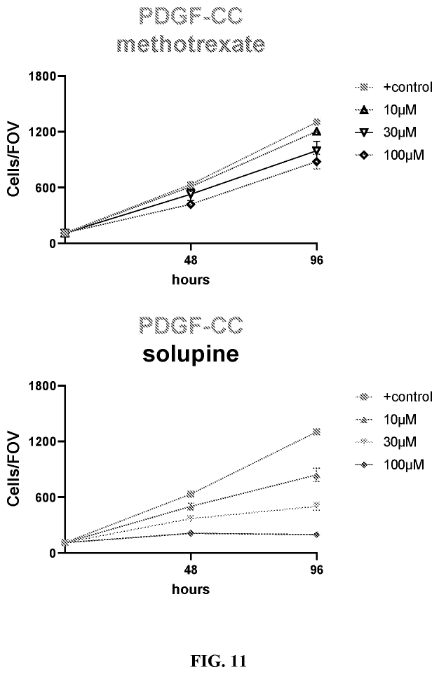

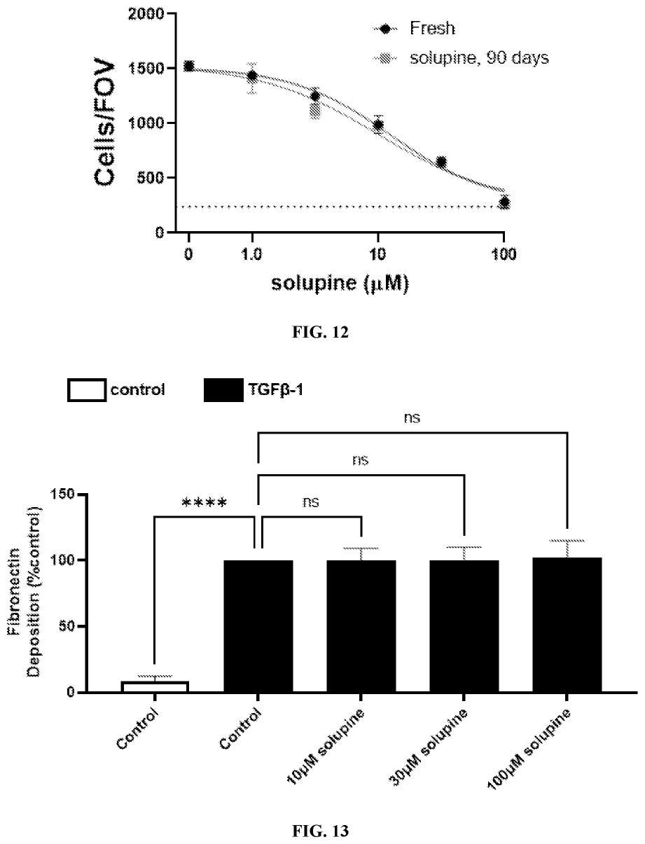

FIG. 12 shows results of la study of solupine functional stability. A powder stock of solupine was dissolved in saline and stored in an opaque, sealed container at 4° C. for 90 days. The potency and efficacy of this stored stock was then compared to freshly solubilized stock of solupine dissolved in saline. ARPE-19 cells were cultured for 4 days with FGF-β and the indicated concentration of fresh or 90-day-old solupine stock. Cells were then assayed for proliferation as described herein for FIGS. 10 and 11. N=3. Results: solupine stock is functionally stable for at least 90 days under these conditions.

### Example 6

Effect of methotrexate, solupine, sertindole, penfluridol, pimozide, and fluspirilene, on FGF-β stimulated proliferation was determined. Methods: ARPE-19 cells were seeded into 96-well plates (1,000 cells/well) and incubated for 6 h at 37° C. After, media was removed and exchanged for media containing 0% fetal bovine serum+/−10 ng/mL FGF-β, and the indicated concentration of compound. Compound concentrations were selected based on the potencies observed in FIGS. 4A-6B, and 9A-9C. After the indicated number of hours, cells were fixed in 4% formalin, and DAPI stained. Cells were then imaged using an automated microscope (Biotek Cytation5) and the number of DAPI nuclei were quantified using onboard software (Gen5). Raw cells/field of view are plotted. N=3. Results: All tested D2 antagonists were more potent than methotrexate at blocking FGF-β stimulated proliferation. FGF-β levels are elevated in patients with PVR. Results are shown in FIGS. 10A and 10B.

### Example 7

Effect of methotrexate and solupine on PDGF-CC stimulated proliferation was determined. Methods: experiments were carried out as described in Example 6, except 10 ng/mL PDGF-CC was used to stimulate proliferation. Results: Solupine is more potent than methotrexate at blocking PDGF-BB stimulated proliferation. PDGF-CC levels are elevated in patients with PVR. Results are shown in FIG. 11.

### Example 8

FIG. 13 shows that solupine does not block fibronectin deposition by conjunctival fibroblasts. Human conjunctival fibroblasts were cultured under the same conditions described in FIGS. 4-6 and fibronectin deposition was measured after treating with the indicated concentrations of solupine. N=3. Results: solupine does not regulate ECM deposition of conjunctival fibroblasts.

FIG. 15 shows that solupine blocks fibronectin deposition in ARPE-19 cells stimulated with a cocktail of profibrotic ligands. ARPE-19 cells were cultured as described in FIGS. 4-6 and fibronectin was measured after stimulating the cells with a combination of profibrotic soluble factors including: T1=TGF-β1, T2=TGF-β2, Tα=TNF-α, Fβ=FGF-β, PC=PDGF-CC, (all at 10 ng/mL). +/−10 μM solupine. N=3. Results: solupine maintains its efficacy at blocking RPE cell fibronectin deposition when profibrotic factors are combined in a cocktail.

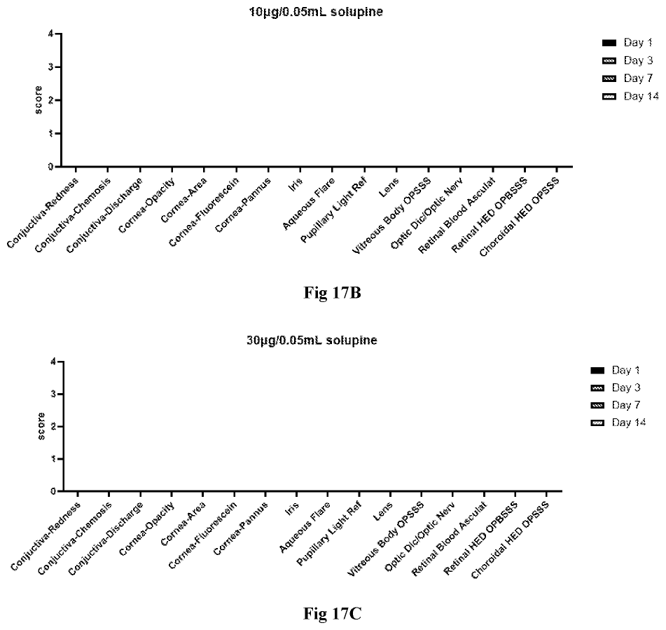

FIG. 17 shows toxicology scoring of mouse eye injected with solupine. Mice were injected with 1 μL of PBS (FIG. 17A), 1 μL PBS containing 10 μg/0.05 mL solupine (FIG. 17B), or 1 μL PBS containing 30 μg/0.05 mL solupine (FIG. 17C). The eyes were then analyzed by a pathologist and scored for the indicated observations of ocular toxicities. over the course of 14 days. Work was performed under contract with Labcorp. N=3-6 mice/group. Results: No observable toxicity associated with either dose of solupine.

### Example 9

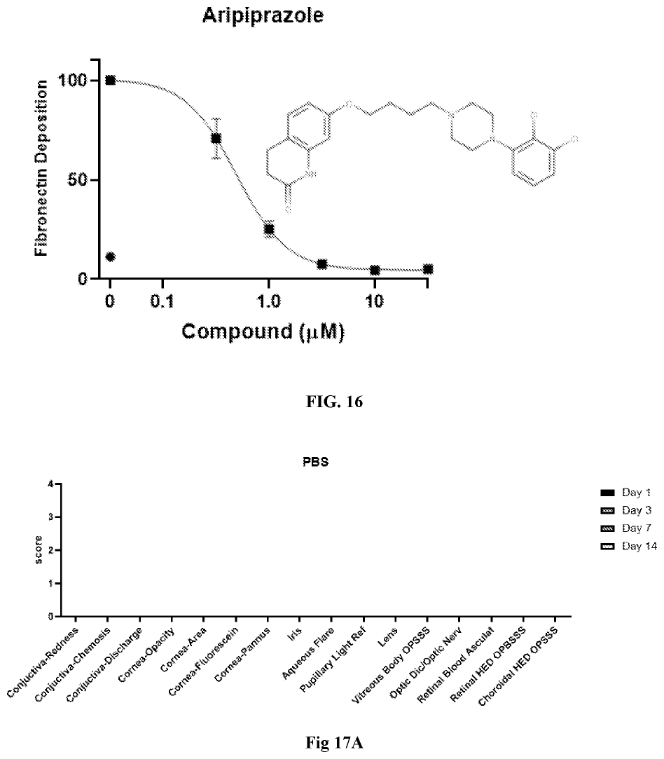

FIG. 16 shows that aripiprazole blocks fibronectin deposition. Experiment performed identically to FIGS. 4-6. N=3.

### REFERENCES

1. Beaulieu, J. M., and Gainetdinov, R. R. (2011). The Physiology, Signaling, and Pharmacology of Dopamine Receptors. Pharmacol Rev 63, 182-217.
2. Bell, J. L., Haak, A. J., Wade, S. M., Kirchhoff, P. D., Neubig, R. R., and Larsen, S. D. (2013). Optimization of novel nipecotic bis(amide) inhibitors of the Rho/MKL1/SRF transcriptional pathway as potential anti-metastasis agents. Bioorg Med Chem Lett 23, 3826-3832.
3. Boyd, K. N., and Mailman, R. B. (2012). Dopamine receptor signaling and current and future antipsychotic drugs. Handb Exp Pharmacol, 53-86.
4. Breslin, S., and O'Driscoll, L. (2013). Three-dimensional cell culture: the missing link in drug discovery. Drug Discov Today 18, 240-249.
5. Brogden, R. N., and Markham, A. (1997). Fenoldopam: a review of its pharmacodynamic and pharmacokinetic properties and intravenous clinical potential in the management of hypertensive urgencies and emergencies. Drugs 54, 634-650.
6. Choi, K. M., Haak, A. J., Diaz Espinosa, A. M., Cummins, K. A., Link, P. A., Aravamudhan, A., Wood, D. K., and Tschumperlin, D. J. (2021). GPCR-mediated YAP/TAZ inactivation in fibroblasts via EPAC1/2, RAP2C, and MAP4K7. J Cell Physiol 236, 7759-7774.
7. Diaz-Espinosa, A. M., Link, P. A., Sicard, D., Jorba, I., Tschumperlin, D. J., and Haak, A. J. (2020). Dopamine D1 receptor stimulates cathepsin K-dependent degradation and resorption of collagen I in lung fibroblasts. J Cell Sci 133.
8. Dong, F., An, J. H., Ren, Y. P., Yan, D. S., Zhou, X. T., Lu, F., Hu, D. N., Chen, J. F., and Qu, J. (2007). [Expression of dopamine receptor D2 and adenosine receptor A2A in human retinal pigment epithelium]. Zhonghua Yan Ke Za Zhi 43, 1110-1113.
9. Friedlander, M. (2007). Fibrosis and diseases of the eye. J Clin Invest 117, 576-586.
10. Gray, D. L., Allen, J. A., Mente, S., O'Connor, R. E., DeMarco, G. J., Efremov, I., Tierney, P., Volfson, D., Davoren, J., Guilmette, E., et al. (2018). Impaired beta-arrestin recruitment and reduced desensitization by non-catechol agonists of the D1 dopamine receptor. Nat Commun 9, 674.
11. Guha, S., Baltazar, G. C., Tu, L. A., Liu, J., Lim, J. C., Lu, W., Argall, A., Boesze-Battaglia, K., Laties, A. M., and Mitchell, C. H. (2012). Stimulation of the D5 dopamine receptor acidifies the lysosomal pH of retinal pigmented epithelial cells and decreases accumulation of autofluorescent photoreceptor debris. J Neurochem 122, 823-833.
12. Haak, A. J., Ducharme, M. T., Espinosa, A. M. D., and Tschumperlin, D. J. (2020).

Targeting GPCR Signaling for Idiopathic Pulmonary Fibrosis Therapies. Trends in Pharmacological Sciences 41, 172-182.
13. Haak, A. J., Kostallari, E., Sicard, D., Ligresti, G., Choi, K. M., Caporarello, N., Jones, D. L., Tan, Q., Meridew, J., Diaz Espinosa, A. M., et al. (2019). Selective YAP/TAZ inhibition in fibroblasts via dopamine receptor D1 agonism reverses fibrosis. Sci Transl Med 11.
14. Hazim, R. A., Volland, S., Yen, A., Burgess, B. L., and Williams, D. S. (2019).

Rapid differentiation of the human RPE cell line, ARPE-19, induced by nicotinamide. Exp Eye Res 179, 18-24.
15. Idrees, S., Sridhar, J., and Kuriyan, A. E. (2019). Proliferative Vitreoretinopathy: A Review. Int Ophthalmol Clin 59, 221-240.
16. Insel, P. A., Murray, F., Yokoyama, U., Romano, S., Yun, H., Brown, L., Snead, A., Lu, D., and Aroonsakool, N. (2012). cAMP and Epac in the regulation of tissue fibrosis. Br J Pharmacol 166, 447-456.
17. Jiang, X., Shao, M., Liu, X., Liu, X., Zhang, X., Wang, Y., Yin, K., Wang, S., Hu, Y., Jose, P. A., et al. (2021a). Reversible Treatment of Pressure Overload-Induced Left Ventricular Hypertrophy through Drd5 Nucleic Acid Delivery Mediated by Functional Polyaminoglycoside. Adv Sci (Weinh) 8, 2003706.
18. Jiang, X. L., Shao, M. Y., Liu, X., Liu, X., Zhang, X., Wang, Y. M., Yin, K. L., Wang, S. Y., Hu, Y., Jose, P. A., et al. (2021b). Reversible Treatment of Pressure Overload-Induced Left Ventricular Hypertrophy through Drd5 Nucleic Acid Delivery Mediated by Functional Polyaminoglycoside. Adv Sci 8.
19. Kirchhof, B., and Sorgente, N. (1989). Pathogenesis of proliferative vitreoretinopathy. Modulation of retinal pigment epithelial cell functions by vitreous and macrophages. Dev Ophthalmol 16, 1-53.
20. Kita, T., Hata, Y., Arita, R., Kawahara, S., Miura, M., Nakao, S., Mochizuki, Y., Enaida, H., Goto, Y., Shimokawa, H., et al. (2008). Role of TGF-beta in proliferative vitreoretinal diseases and ROCK as a therapeutic target. P Natl Acad Sci USA 105, 17504-17509.
21. Martucci, A., Cesareo, M., Pinazo-Duran, M. D., Di Pierro, M., Di Marino, M., Nucci, C., Coletta, M., and Mancino, R. (2020). Is there a relationship between dopamine and rhegmatogenous retinal detachment?Neural Regen Res 15, 311-314.
22. Miller, C. G., Budoff, G., Prenner, J. L., and Schwarzbauer, J. E. (2017). Minireview: Fibronectin in retinal disease. Exp Biol Med (Maywood) 242, 1-7.
23. Ming, M., Li, X. P., Fan, X. L., Yang, D. H., Li, L., Chen, S., Gu, Q., and Le, W. D. (2009). Retinal pigment epithelial cells secrete neurotrophic factors and synthesize dopamine: possible contribution to therapeutic effects of RPE cell transplantation in Parkinson's disease. J Transl Med 7.
24. Mou, Y., Liu, J., Pan, T., Wang, Q., Miao, K., Xu, Y., Xiong, W., and Yu, J. (2021). Dopamine receptor agonists ameliorate bleomycin-induced pulmonary fibrosis by repressing fibroblast differentiation and proliferation. Biomed Pharmacother 139, 111500.
25. Mudhar, H. S. (2020). A brief review of the histopathology of proliferative vitreoretinopathy (PVR). Eye (Lond) 34, 246-250.
26. Orita, T., Kimura, K., and Sonoda, K. (2013). Effect of prostaglandin E2 on collagen gel contraction in mouse retinal pigment epithelium cells. Invest Ophth Vis Sci 54.
27. Popovic, D., Nuss, P., and Vieta, E. (2015). Revisiting loxapine: a systematic review. Ann Gen Psychiatry 14, 15.
28. Qing, J., Ren, Y., Zhang, Y., Yan, M., Zhang, H., Wu, D., Ma, Y., Chen, Y., Huang, X., Wu, Q., et al. (2021). Dopamine receptor D2 antagonization normalizes profibrotic macrophage-endothelial crosstalk in non-alcoholic steatohepatitis. J Hepatol.
29. Roy, S., Amin, S., and Roy, S. (2016). Retinal fibrosis in diabetic retinopathy. Exp Eye Res 142, 71-75.
30. Seagle, B. L. L., Rezai, K. A., Kobori, Y., Gasyna, E. M., Rezaei, K. A., and Norris, J. R. (2005). Melanin photoprotection in the human retinal pigment epithelium and its correlation with light-induced cell apoptosis. P Natl Acad Sci USA 102, 8978-8983.
31. Sparrow, J. R., Hicks, D., and Hamel, C. P. (2010). The retinal pigment epithelium in health and disease. Curr Mol Med 10, 802-823.
32. Sriram, K., and Insel, P. A. (2018). G Protein-Coupled Receptors as Targets for Approved Drugs: How Many Targets and How Many Drugs?Mol Pharmacol 93, 251-258.
33. Tamiya, S., and Kaplan, H. J. (2016). Role of epithelial-mesenchymal transition in proliferative vitreoretinopathy. Exp Eye Res 142, 26-31.
34. Yin, F., Tian, Z. M., Liu, S., Zhao, Q. J., Wang, R. M., Shen, L., Wieman, J., and Yan, Y. (2012). Transplantation of Human Retinal Pigment Epithelium Cells in the Treatment for Parkinson Disease. Cns Neurosci Ther 18, 1012-1020.
35. Zhao, B., Li, S., Guo, Z., Chen, Z., Zhang, X., Xu, C., Chen, J., and Wei, C. (2021). Dopamine receptor D2 inhibition alleviates diabetic hepatic stellate cells fibrosis by regulating the TGF-beta1/Smads and NFkappaB pathways. Clin Exp Pharmacol Physiol 48, 370-380.

### Numbered Paragraphs

In some embodiments, the invention can be described by reference to the following numbered paragraphs.

Paragraph 1. A method of treating or preventing an ocular fibrotic pathology, the method comprising administering to a subject in need thereof a therapeutically effective amount of a dopamine receptor D2 (DRD2) antagonist, or a pharmaceutically acceptable salt thereof.

Paragraph 2. The method of paragraph 1, wherein the ocular fibrotic pathology is selected from: proliferative vitreoretinopathy (PVR), diabetic retinopathy, ischemic retinopathy, age-related macular degeneration (ARMD), dry ARMD, neovascular ARMD, retinal neovascularization, choroidal neovascularization, epiretinal membrane, keratitis, pterygia, pingueculae, retinopathy of prematurity, glaucoma (including neovascular glaucoma, open-angle glaucoma, angle-closure glaucoma, secondary glaucoma, and childhood glaucoma), Stargardt's disease, sickle cell retinopathy, radiation retinopathy, optic neuropathy, retinal detachment, retinal degeneration, uveitis, dry eye disease, congenital fibrosis of the extraocular muscles (CFEOM), and corneal fibrosis.

Paragraph 3. The method of paragraph 2, wherein the ocular fibrotic pathology is proliferative vitreoretinopathy (“PVR”).

Paragraph 4. The method of paragraph 1, wherein the ocular fibrotic pathology is selected from: opacification and fibrosis of the posterior capsule of the lens following eye surgery, fibrosis following glaucoma filtration surgery, fibrosis following a wound or trauma, conjunctival fibrosis or subconjunctival fibrosis, fibrosis of the ocular muscles, Graves disease, fibrosis following wound healing of the skin around the eye and face, fibrosis of the surface of the eye with pterygium or pingueculae, fibrosis due to choroidal neovascularization and angiogenesis, fibrosis following a corneal wound, fibrosis following corneal laser surgery, fibrosis following refractive surgery, and fibrosis following a corneal transplant.

Paragraph 4a. The method of paragraph 1, wherein the ocular fibrotic pathology is selected from: cataract, ocular melanoma, conjunctival melanoma, retinoblastoma, optic neuritis, ocular cicatricial pemphigoid, ocular surface squamous neoplasia, keratoconus, corneal dystrophies, anterior basement membrane dystrophy, Salzmann's nodular degeneration, corneal diseases, scleritis, Fuch's endothelial corneal dystrophy, ocular lymphoma, myopia, strabismus, nystagmus, corneal haze, corneal scarring, corneal neovascularization, lacrimal gland tumors, primary open angle glaucoma, juvenile glaucoma, angle closure glaucoma, exfoliation glaucoma, and optic nerve disorders.

Paragraph 5. The method of any one of paragraphs 1-4, wherein the administering of the compound comprises administering the compound to the subject by an ocular route.

Paragraph 6. The method of paragraph 5, wherein the ocular route is selected from: intraocular, periocular, subtenon, retrobulbar, intravitreal, intraocular, intracameral, subconjunctival, subtenon, intracorneal, intrastromal, trans-scleral, and suprachoroidal route.

Paragraph 7. The method of paragraph 5, wherein the ocular route comprises a local injection into or about cornea, choroid, retina, vitreous, anterior chamber, sclera, suprachoroidal space, uvea, orbit, eyelid, conjunctiva, or iris.

Paragraph 8. The method of paragraph 5 or 6, wherein the administering of the compound comprises administering the compound in a pharmaceutical formulation selected from: eye-drops, eye ointment, and eye emulsion.

Paragraph 9. A method of inhibiting epithelial to mesenchymal transition (EMT) in a retinal pigment epithelial (RPE) cell, the method comprising contacting the cell with an effective amount of a dopamine receptor D2 (DRD2) antagonist, or a pharmaceutically acceptable salt thereof.

Paragraph 10. A method of inhibiting migration or proliferation of a retinal pigment epithelial (RPE) cell, the method comprising contacting the cell with an effective amount of a dopamine receptor D2 (DRD2) antagonist, or a pharmaceutically acceptable salt thereof.

Paragraph 11. A method of inhibiting expression of a profibrotic gene in a retinal pigment epithelial (RPE) cell, the method comprising contacting the cell with an effective amount of a dopamine receptor D2 (DRD2) antagonist, or a pharmaceutically acceptable salt thereof.

Paragraph 12. The method of paragraph 11, wherein profibrotic gene is selected from Acta2 (α-smooth muscle actin, αSMA), Ctgf (Connective tissue growth factor), Fn1 (Fibronectin), Col1a1 (Collagen I), Col1a2 (Collagen II), and Col3a1 (Collagen III), or any combination thereof.

Paragraph 13. A method of inhibiting extra-cellular matrix production and deposition by a retinal pigment epithelial (RPE) cell, the method comprising contacting the cell with an effective amount of a dopamine receptor D2 (DRD2) antagonist, or a pharmaceutically acceptable salt thereof.

Paragraph 14. A method of enhancing extra-cellular matrix degradation by a retinal pigment epithelial (RPE) cell, the method comprising contacting the cell with an effective amount of a dopamine receptor D2 (DRD2) antagonist, or a pharmaceutically acceptable salt thereof.

Paragraph 15. The method of any one of paragraphs 9-14, wherein the contacting is carried out in vitro, in vivo, or ex vivo.

Paragraph 16. The method of any one of paragraphs 1-15, wherein the dopamine receptor D2 (DRD2) antagonist is selected from loxapine, clozapine, amoxapine, olanzapine, N-desmethyl olanzapine, quetiapine, N-desmethyl clozapine, 8-OH-loxapine, pizotifen, asenapine, and blonanserin, or a pharmaceutically acceptable salt thereof.

Paragraph 17. The method of any one of paragraphs 1-15, wherein the dopamine receptor D2 (DRD2) antagonist is selected from domperidone, pimozide, and sertindole, or a pharmaceutically acceptable salt thereof.

Paragraph 18. The method of any one of paragraphs 1-15, wherein the dopamine receptor D2 (DRD2) antagonist is selected from prochlorperazine, trifluoperizine, and perphenazine, or a pharmaceutically acceptable salt thereof.

Paragraph 19. The method of any one of paragraphs 1-15, wherein the dopamine receptor D2 (DRD2) antagonist is selected from eticlopride, sulpiride, remoxipride, amisulpride, and raclopride, or a pharmaceutically acceptable salt thereof.

Paragraph 20. The method of any one of paragraphs 1-15, wherein the dopamine receptor D2 (DRD2) antagonist is selected from methotrexate, spiperone, fluspirilene, penfluridol, droperidol, timiperone, benperidol, aripiprazole, and lurasidone, or a pharmaceutically acceptable salt thereof.

Paragraph 21. A compound of Formula (I):

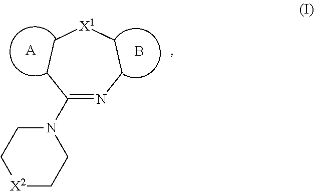

or a pharmaceutically acceptable salt thereof, wherein:
X¹ is selected from O, S, CH₂, and NR^{N};
X² is selected from O, S, and NR^{N};
R^{N} is selected from H, C₁₋₃ alkyl, and C₁₋₃ haloalkyl;
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
provided that at least one of ring A and ring B is 5-6-membered heteroaryl.

Paragraph 22. The compound of paragraph 21, wherein the compound of Formula (I) has formula:

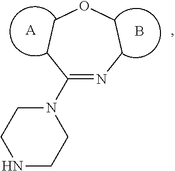

or a pharmaceutically acceptable salt thereof.

Paragraph 23. The compound of paragraph 21, wherein the compound of Formula (I) has formula:

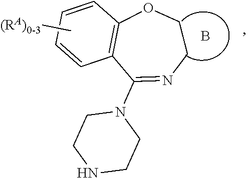

or a pharmaceutically acceptable salt thereof, wherein ring B is 5-6-membered heteroaryl which is optionally substituted with 1, 2, or 3 substituents independently selected from R^{B}.

Paragraph 24. The compound of any one of paragraphs 21-23, wherein ring B is 5-membered heteroaryl which is optionally substituted with R^{B}.

Paragraph 25. The compound of paragraph 24, wherein the 5-membered heteroaryl is selected from thienyl, furyl, and pyrrolyl.

Paragraph 26. The compound of paragraph 24, wherein the 5-membered heteroaryl is thienyl.

Paragraph 27. The compound of any one of paragraphs 21-23, wherein ring B is 6-membered heteroaryl which is optionally substituted with R^{B}.

Paragraph 28. The compound of paragraph 27, wherein the 6-membered heteroaryl is selected from pyridinyl, pyrazinyl, and pyrimidinyl.

Paragraph 29. The compound of paragraph 27, wherein the 6-membered heteroaryl is pyridinyl.

Paragraph 30. The compound of paragraph 21, wherein the compound of Formula (I) has formula:

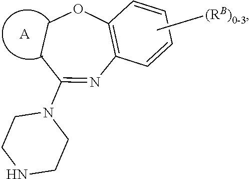

or a pharmaceutically acceptable salt thereof, wherein ring A is 5-6-membered heteroaryl which is optionally substituted with 1, 2, or 3 substituents independently selected from R^{A}.

Paragraph 31. The compound of any one of paragraphs 21-30, wherein ring A is 5-membered heteroaryl which is optionally substituted with R^{A}.

Paragraph 32. The compound of paragraph 31, wherein the 5-membered heteroaryl is selected from thienyl, furyl, and pyrrolyl.

Paragraph 33. The compound of paragraph 31, wherein the 5-membered heteroaryl is thienyl.

Paragraph 34. The compound of any one of paragraphs 21-30, wherein ring B is 6-membered heteroaryl which is optionally substituted with R^{B}.

Paragraph 35. The compound of paragraph 34, wherein the 6-membered heteroaryl is selected from pyridinyl, pyrazinyl, and pyrimidinyl.

Paragraph 36. The compound of paragraph 34, wherein the 6-membered heteroaryl is pyridinyl.

Paragraph 37. The compound of any one of paragraphs 21-36, wherein R^{A} is selected from OH, C₁₋₃ alkoxy, SH, NH₂, C₁₋₃ alkylamino, and di(C₁₋₃ alkyl)amino.

Paragraph 38. The compound of paragraph 37, wherein R^{A} is selected from OH, C₁₋₃ alkoxy, and NH₂.

Paragraph 39. The compound of any one of paragraphs 21-38, wherein R^{B} is selected from OH, C₁₋₃ alkoxy, SH, NH₂, C₁₋₃ alkylamino, and di(C₁₋₃ alkyl)amino.

Paragraph 40. The compound of paragraph 39, wherein R^{B} is selected from OH, C₁₋₃ alkoxy, and NH₂.

Paragraph 41. The compound of paragraph 21, wherein the compound of Formula (I) is selected from:

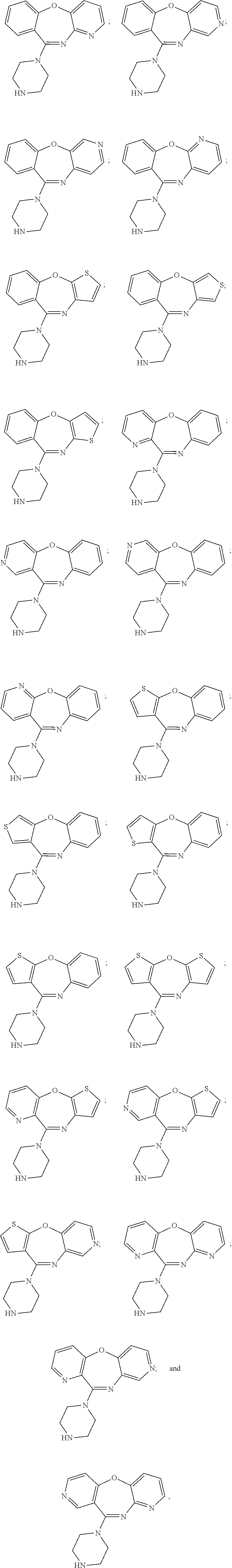

or a pharmaceutically acceptable salt thereof.

Paragraph 42. A compound of Formula (II):

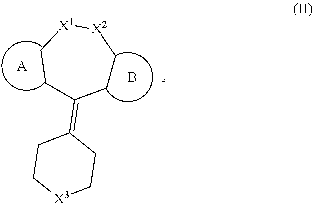

or a pharmaceutically acceptable salt thereof, wherein:
X¹ is selected from O, S, CH₂, and NR^{N};
X² is selected from O, S, CH₂, and NR^{N};
X³ is selected from O, S, and NR^{N};
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino.

Paragraph 43. The compound of paragraph 42, wherein the compound of Formula (II) has formula:

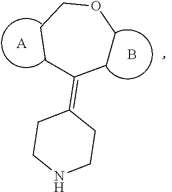

or a pharmaceutically acceptable salt thereof.

Paragraph 44. The compound of paragraph 42, wherein the compound of Formula (II) has formula:

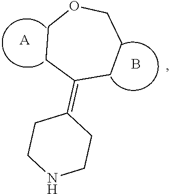

or a pharmaceutically acceptable salt thereof.

Paragraph 45. The compound of paragraph 42, wherein the compound of Formula (II) has formula:

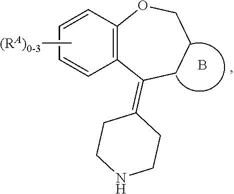

or a pharmaceutically acceptable salt thereof, wherein B is 5-6-membered heteroaryl which is optionally substituted with 1, 2, or 3 substituents independently selected from R^{B}.

Paragraph 46. The compound of paragraph 42, wherein the compound of Formula (II) has formula:

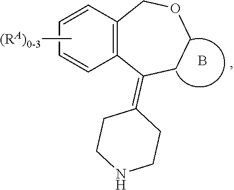

or a pharmaceutically acceptable salt thereof, B is 5-6-membered heteroaryl which is optionally substituted with 1, 2, or 3 substituents independently selected from R^{B}.

Paragraph 47. The compound of any one of paragraphs 42-46, wherein ring B is 5-membered heteroaryl which is optionally substituted with R^{B}.

Paragraph 48. The compound of paragraph 47, wherein the 5-membered heteroaryl is selected from thienyl, furyl, and pyrrolyl.

Paragraph 49. The compound of paragraph 47, wherein the 5-membered heteroaryl is thienyl.

Paragraph 50. The compound of any one of paragraphs 42-46, wherein ring B is 6-membered heteroaryl which is optionally substituted with R^{B}.

Paragraph 51. The compound of paragraph 50, wherein the 6-membered heteroaryl is selected from pyridinyl, pyrazinyl, and pyrimidinyl.

Paragraph 52. The compound of paragraph 50, wherein the 6-membered heteroaryl is pyridinyl.

Paragraph 53. The compound of paragraph 42, wherein the compound of Formula (II) has formula:

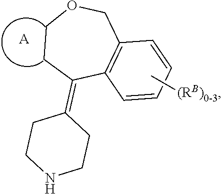

or a pharmaceutically acceptable salt thereof, wherein ring A is 5-6-membered heteroaryl which is optionally substituted with 1, 2, or 3 substituents independently selected from R^{A}.

Paragraph 54. The compound of paragraph 42, wherein the compound of Formula (II) has formula:

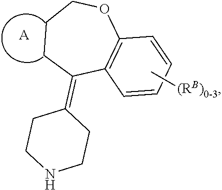

or a pharmaceutically acceptable salt thereof, wherein ring A is 5-6-membered heteroaryl which is optionally substituted with 1, 2, or 3 substituents independently selected from R^{A}.

Paragraph 55. The compound of any one of paragraphs 42-54, wherein ring A is 5-membered heteroaryl which is optionally substituted with R^{A}.

Paragraph 56. The compound of paragraph 55, wherein the 5-membered heteroaryl is selected from thienyl, furyl, and pyrrolyl.

Paragraph 57. The compound of paragraph 55, wherein the 5-membered heteroaryl is thienyl.

Paragraph 58. The compound of any one of paragraphs 42-54, wherein ring B is 6-membered heteroaryl which is optionally substituted with R^{B}.

Paragraph 59. The compound of paragraph 58, wherein the 6-membered heteroaryl is selected from pyridinyl, pyrazinyl, and pyrimidinyl.

Paragraph 60. The compound of paragraph 58, wherein the 6-membered heteroaryl is pyridinyl.

Paragraph 61. The compound of any one of paragraphs 42-60, wherein R^{A} is selected from OH, C₁₋₃ alkoxy, SH, NH₂, C₁₋₃ alkylamino, and di(C₁₋₃ alkyl)amino.

Paragraph 62. The compound of paragraph 61, wherein R^{A} is selected from OH, C₁₋₃ alkoxy, and NH₂.

Paragraph 63. The compound of any one of paragraphs 42-62, wherein R^{B} is selected from OH, C₁₋₃ alkoxy, SH, NH₂, C₁₋₃ alkylamino, and di(C₁₋₃ alkyl)amino.

Paragraph 64. The compound of paragraph 63, wherein R^{B} is selected from OH, C₁₋₃ alkoxy, and NH₂.

Paragraph 65. The compound of paragraph 42, wherein the compound of Formula (II) is selected from:

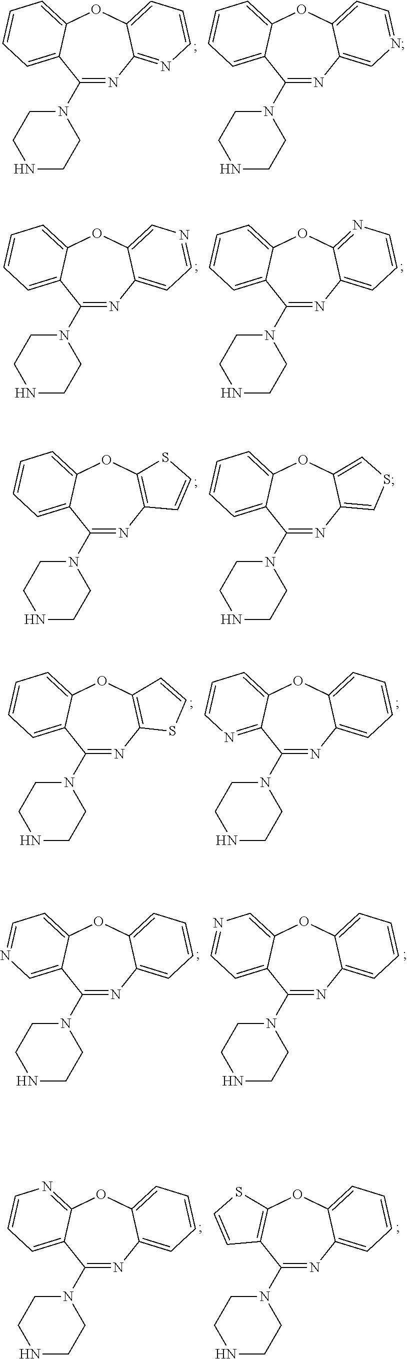

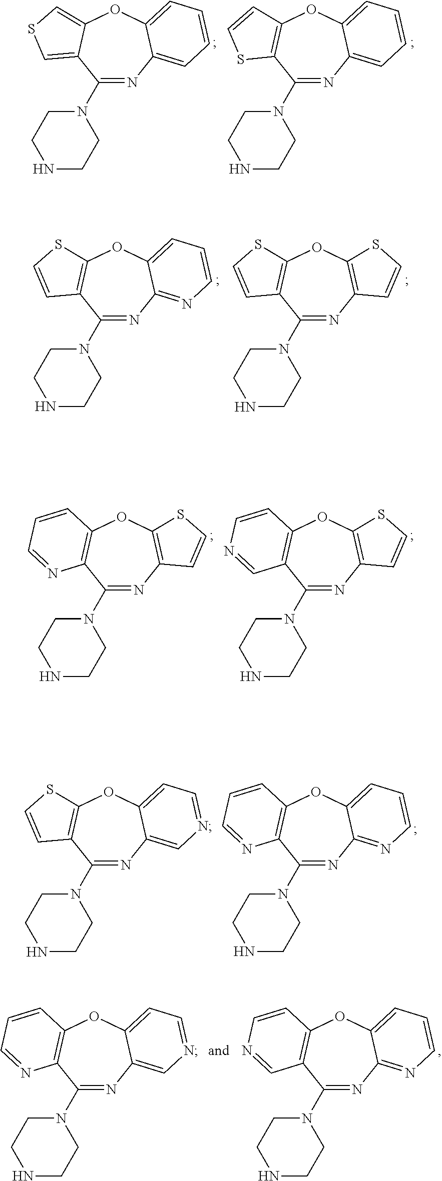

or a pharmaceutically acceptable salt thereof.

Paragraph 66. A pharmaceutical composition comprising a compound of any one of paragraphs 21-65, or a pharmaceutically acceptable salt thereof, and a pharmaceutically acceptable carrier.

Paragraph 67. A method of treating or preventing an ocular fibrotic pathology, the method comprising administering to a subject in need thereof a therapeutically effective amount of a compound of any one of paragraphs 21-65, or a pharmaceutically acceptable salt thereof.

Paragraph 68. The method of paragraph 67, wherein the ocular fibrotic pathology is selected from: proliferative vitreoretinopathy (PVR), diabetic retinopathy, ischemic retinopathy, age-related macular degeneration (ARMD), dry ARMD, neovascular ARMD, keratitis, pterygia, pingueculae, retinopathy of prematurity, glaucoma (including neovascular glaucoma, open-angle glaucoma, angle-closure glaucoma, secondary glaucoma, and childhood glaucoma), Stargardt's disease, sickle cell retinopathy, radiation retinopathy, optic neuropathy, retinal detachment, retinal degeneration, uveitis, dry eye disease, congenital fibrosis of the extraocular muscles (CFEOM), and corneal fibrosis.

Paragraph 69. The method of paragraph 68, wherein the ocular fibrotic pathology is proliferative vitreoretinopathy (“PVR”).

Paragraph 70. The method of paragraph 67, wherein the ocular fibrotic pathology is selected from: opacification and fibrosis of the posterior capsule of the lens following eye surgery, fibrosis following glaucoma filtration surgery, fibrosis following a wound or trauma, conjunctival fibrosis or subconjunctival fibrosis, fibrosis of the ocular muscles, Graves disease, fibrosis following wound healing of the skin around the eye and face, fibrosis of the surface of the eye with pterygium or pingueculae, fibrosis due to choroidal neovascularization and angiogenesis, fibrosis following a corneal wound, fibrosis following corneal laser surgery, fibrosis following refractive surgery, and fibrosis following a corneal transplant.

Paragraph 70a. The method of paragraph 67, wherein the ocular fibrotic pathology is selected from: cataract, ocular melanoma, conjunctival melanoma, retinoblastoma, optic neuritis, ocular cicatricial pemphigoid, ocular surface squamous neoplasia, keratoconus, corneal dystrophies, anterior basement membrane dystrophy, Salzmann's nodular degeneration, corneal diseases, scleritis, Fuch's endothelial corneal dystrophy, ocular lymphoma, myopia, strabismus, nystagmus, corneal haze, corneal scarring, corneal neovascularization, lacrimal gland tumors, primary open angle glaucoma, juvenile glaucoma, angle closure glaucoma, exfoliation glaucoma, and optic nerve disorders.

Paragraph 71. The method of any one of paragraphs 67-70, wherein the administering of the compound comprises administering the compound to the subject by an ocular route.

Paragraph 72. The method of paragraph 71, wherein the ocular route is selected from: intravitreal, intraocular, intracameral, subconjunctival, subtenon, intracorneal, intrastromal, trans-scleral, and suprachoroidal route.

Paragraph 73. The method of paragraph 71, wherein the ocular route comprises a local injection into or about cornea, choroid, retina, vitreous, uvea, orbit, eyelid, conjunctiva, or iris.

Paragraph 74. The method of paragraph 71, wherein the administering of the compound comprises administering the compound in a pharmaceutical formulation selected from: eye-drops, eye ointment, and eye emulsion.

Paragraph 75. A method of inhibiting epithelial to mesenchymal transition (EMT) in a retinal pigment epithelial (RPE) cell, the method comprising contacting the cell with an effective amount of a compound of any one of paragraphs 21-65, or a pharmaceutically acceptable salt thereof.

Paragraph 76. A method of inhibiting migration or proliferation of a retinal pigment epithelial (RPE) cell, the method comprising contacting the cell with an effective amount of a compound of any one of paragraphs 21-65, or a pharmaceutically acceptable salt thereof.

Paragraph 77. A method of inhibiting expression of a profibrotic gene in a retinal pigment epithelial (RPE) cell, the method comprising contacting the cell with an effective amount of a compound of any one of paragraphs 21-65, or a pharmaceutically acceptable salt thereof.

Paragraph 78. The method of paragraph 77, wherein profibrotic gene is selected from Acta2 (α-smooth muscle actin, αSMA), Ctgf (Connective tissue growth factor), Fn1 (Fibronectin), Col1a1 (Collagen I), Col1a2 (Collagen II), and Col3a1 (Collagen III), or any combination thereof.

Paragraph 79. A method of inhibiting extra-cellular matrix production and deposition by a retinal pigment epithelial (RPE) cell, the method comprising contacting the cell with an effective amount of a compound of any one of paragraphs 21-65, or a pharmaceutically acceptable salt thereof.

Paragraph 80. A method of enhancing extra-cellular matrix degradation by a retinal pigment epithelial (RPE) cell, the method comprising contacting the cell with an effective amount of a compound of any one of paragraphs 21-65, or a pharmaceutically acceptable salt thereof.

Paragraph 81. The method of any one of paragraphs 75-80, wherein the contacting is carried out in vitro, in vivo, or ex vivo.

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A method of treating or preventing an ocular fibrotic pathology, the method comprising administering to a subject in need thereof a therapeutically effective amount of (a) a dopamine receptor D2 (DRD2) antagonist, or a pharmaceutically acceptable salt thereof, (b) a compound of Formula (I), or a pharmaceutically acceptable salt thereof, or (c) a compound of Formula (II), or a pharmaceutically acceptable salt thereof,
wherein Formula (I) is: 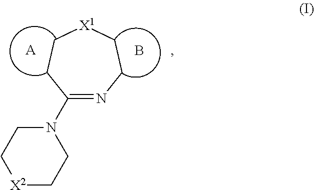
wherein:
X¹ is selected from O, S, CH₂, and NR^{N};
X² is selected from O, S, and NR^{N};
R^{N} is selected from H, C₁₋₃ alkyl, and C₁₋₃ haloalkyl;
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
provided that at least one of ring A and ring B is 5-6-membered heteroaryl; and
wherein Formula (II) is: 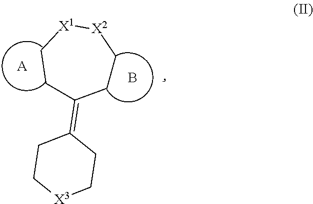
wherein:
X¹ is selected from O, S, CH₂, and NR^{N};
X² is selected from O, S, CH₂, and NR^{N};
X³ is selected from O, S, and NR^{N};
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino.

2. The method of claim 1, wherein the ocular fibrotic pathology is selected from the group consisting of proliferative vitreoretinopathy (PVR), diabetic retinopathy, ischemic retinopathy, age-related macular degeneration (ARMD), dry ARMD, neovascular ARMD, retinal neovascularization, choroidal neovascularization, epiretinal membrane, keratitis, pterygia, pingueculae, retinopathy of prematurity, glaucoma (including neovascular glaucoma, open-angle glaucoma, angle-closure glaucoma, secondary glaucoma, and childhood glaucoma), Stargardt's disease, sickle cell retinopathy, radiation retinopathy, optic neuropathy, retinal detachment, retinal degeneration, uveitis, dry eye disease, congenital fibrosis of the extraocular muscles (CFEOM), and corneal fibrosis.

3. The method of claim 2, wherein the ocular fibrotic pathology is proliferative vitreoretinopathy (“PVR”).

4. The method of claim 1, wherein the ocular fibrotic pathology is selected from the group consisting of opacification and fibrosis of the posterior capsule of the lens following eye surgery, fibrosis following glaucoma filtration surgery, fibrosis following a wound or trauma, conjunctival fibrosis or subconjunctival fibrosis, fibrosis of the ocular muscles, Graves disease, fibrosis following wound healing of the skin around the eye and face, fibrosis of the surface of the eye with pterygium or pingueculae, fibrosis due to choroidal neovascularization and angiogenesis, fibrosis following a corneal wound, fibrosis following corneal laser surgery, fibrosis following refractive surgery, and fibrosis following a corneal transplant.

5. The method of claim 1, wherein the ocular fibrotic pathology is selected from the group consisting of cataract, ocular melanoma, conjunctival melanoma, retinoblastoma, optic neuritis, ocular cicatricial pemphigoid, ocular surface squamous neoplasia, keratoconus, corneal dystrophies, anterior basement membrane dystrophy, Salzmann's nodular degeneration, corneal diseases, scleritis, Fuch's endothelial corneal dystrophy, ocular lymphoma, myopia, strabismus, nystagmus, corneal haze, corneal scarring, corneal neovascularization, lacrimal gland tumors, primary open angle glaucoma, juvenile glaucoma, angle closure glaucoma, exfoliation glaucoma, and optic nerve disorders.

6. A method selected from the group consisting of:
(a) inhibiting epithelial to mesenchymal transition (EMT) in a retinal pigment epithelial (RPE) cell,
(b) inhibiting migration or proliferation of a retinal pigment epithelial (RPE) cell,
(c) inhibiting expression of a profibrotic gene in a retinal pigment epithelial (RPE) cell,
(d) inhibiting extra-cellular matrix production and deposition by a retinal pigment epithelial (RPE) cell, and
(e) enhancing extra-cellular matrix degradation by a retinal pigment epithelial (RPE) cell,
wherein the method comprises contacting the cell with an effective amount of (a) a dopamine receptor D2 (DRD2) antagonist, or a pharmaceutically acceptable salt thereof, (b) a compound of Formula (I), or a pharmaceutically acceptable salt thereof, or (c) a compound of Formula (II), or a pharmaceutically acceptable salt thereof,
wherein Formula (I) is: 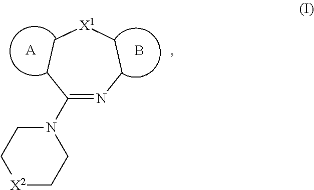
wherein:
X¹ is selected from O, S, CH₂, and NR^{N},
X² is selected from O, S, and NR^{N},
R^{N} is selected from H, C₁₋₃ alkyl, and C₁₋₃ haloalkyl;
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
provided that at least one of ring A and ring B is 5-6-membered heteroaryl; and
wherein Formula (II) is: 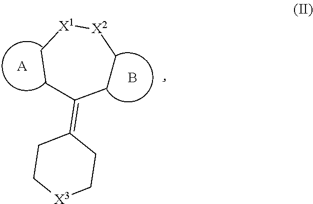
wherein:
X¹ is selected from O, S, CH₂, and NR^{N};
X² is selected from O, S, CH₂, and NR^{N};
X³ is selected from O, S, and NR^{N};
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino.

7. The method of claim 6, wherein the contacting is carried out in vivo.

8. The method of claim 1, wherein said method comprises administering to said subject said therapeutically effective amount of said DRD2 antagonist, wherein said DRD2 antagonist is selected from the group consisting of loxapine, clozapine, amoxapine, olanzapine, N-desmethyl olanzapine, quetiapine, N-desmethyl clozapine, 8-OH-loxapine, pizotifen, asenapine, blonanserin, domperidone, pimozide, sertindole, prochlorperazine, trifluoperizine, perphenazine, eticlopride, sulpiride, remoxipride, amisulpride, raclopride, methotrexate, spiperone, fluspirilene, penfluridol, droperidol, timiperone, benperidol, aripiprazole, and lurasidone, or a pharmaceutically acceptable salt thereof.

9. A compound of Formula (I): 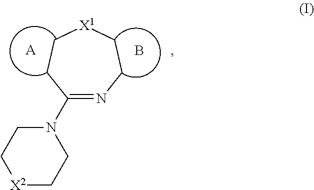
or a pharmaceutically acceptable salt thereof, wherein:
X¹ is selected from O, S, CH₂, and NR^{N};
X² is selected from O, S, and NR^{N};
R^{N} is selected from H, C₁₋₃ alkyl, and C₁₋₃ haloalkyl;
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
provided that at least one of ring A and ring B is 5-6-membered heteroaryl.

10. The compound of claim 9, wherein the compound of Formula (I) has formula: 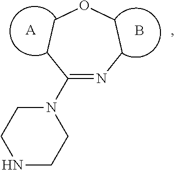
or a pharmaceutically acceptable salt thereof.

11. The compound of claim 9, wherein the compound of Formula (I) is selected from: 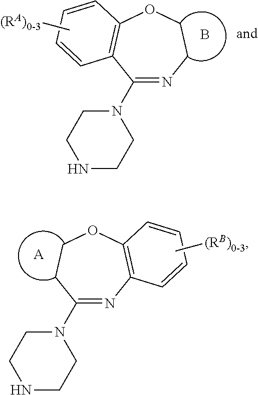
or a pharmaceutically acceptable salt thereof, wherein:
ring A is 5-6-membered heteroaryl which is optionally substituted with 1, 2, or 3 substituents independently selected from R^{A};
each R^{A} is independently selected from OH, C₁₋₃ alkoxy, SH, NH₂, C₁₋₃ alkylamino, and di(C₁₋₃ alkyl)amino; and
ring B is 5-6-membered heteroaryl which is optionally substituted with 1, 2, or 3 substituents independently selected from R^{B};
each R^{B} is independently selected from OH, C₁₋₃ alkoxy, SH, NH₂, C₁₋₃ alkylamino, and di(C₁₋₃ alkyl)amino.

12. The compound of claim 11, wherein:
ring A is selected from thienyl, furyl, and pyrrolyl;
each R^{A} is independently selected from OH, C₁₋₃ alkoxy, and NH₂;
ring B is pyridinyl, pyrazinyl, and pyrimidinyl; and
each R^{B} is selected from OH, C₁₋₃ alkoxy, SH, NH₂, C₁₋₃ alkylamino, and di(C₁₋₃ alkyl)amino.

13. The compound of claim 9, wherein the compound of Formula (I) is selected from the group consisting ofany one of: 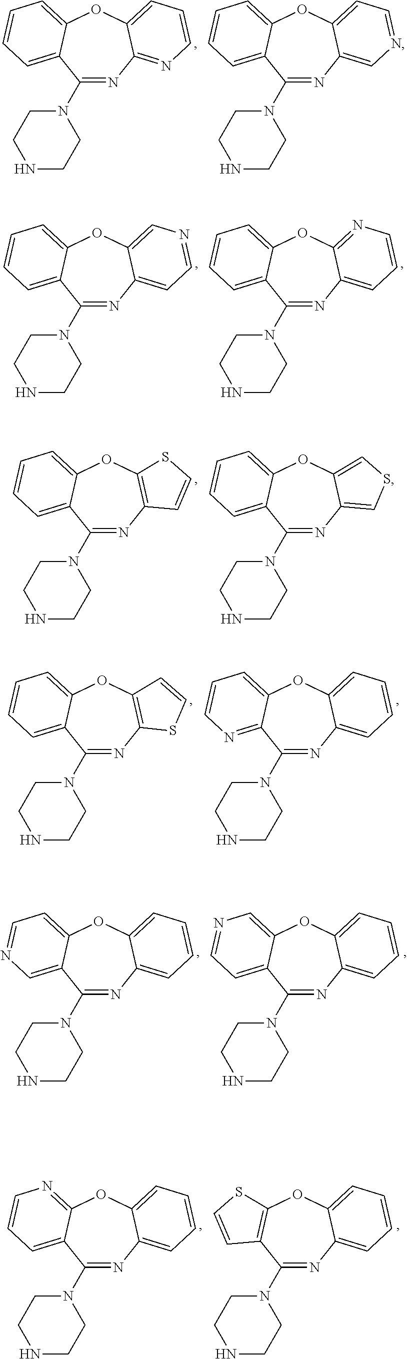 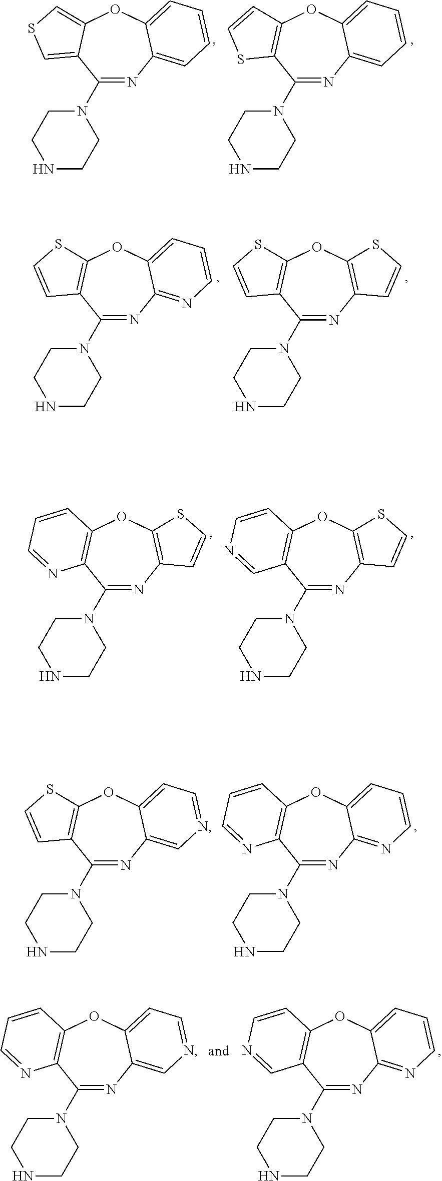
or a pharmaceutically acceptable salt thereof.

14. A compound of Formula (II): 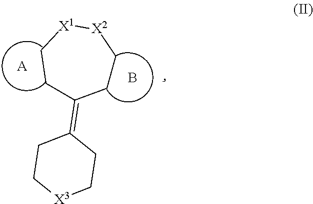
or a pharmaceutically acceptable salt thereof, wherein:
X¹ is selected from O, S, CH₂, and NR^{N};
X² is selected from O, S, CH₂, and NR^{N};
X³ is selected from O, S, and NR^{N};
ring A is selected from phenyl and 5-6-membered heteroaryl which is optionally substituted with 1, 2, or 3 substituents independently selected from R^{A};
each R^{A} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
ring B is selected from phenyl and 5-6-membered heteroaryl which is optionally substituted with 1, 2, or 3 substituents independently selected from R^{B}; and
each R^{B} is independently selected from halo, CN, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino.

15. The compound of claim 14, wherein the compound of Formula (II) is selected from: 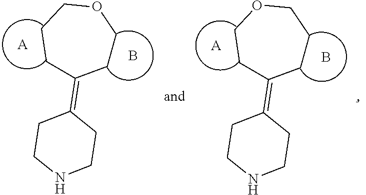
or a pharmaceutically acceptable salt thereof.

16. The compound of claim 15, wherein the compound of Formula (II) is selected from the group consisting of: 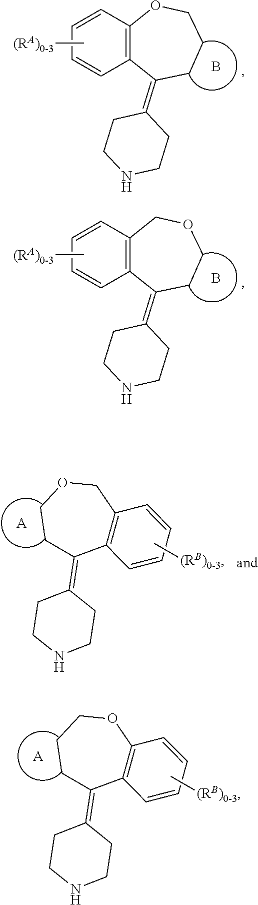
or a pharmaceutically acceptable salt thereof, wherein:
ring A is 5-6-membered heteroaryl which is optionally substituted with 1, 2, or 3 substituents independently selected from R^{A};
each R^{A} is independently selected from OH, C₁₋₃ alkoxy, SH, NH₂, C₁₋₃ alkylamino, and di(C₁₋₃ alkyl)amino; and
ring B is 5-6-membered heteroaryl which is optionally substituted with 1, 2, or 3 substituents independently selected from R^{B};
each R^{B} is independently selected from OH, C₁₋₃ alkoxy, SH, NH₂, C₁₋₃ alkylamino, and di(C₁₋₃ alkyl)amino.

17. The compound of claim 16, wherein:
ring A is selected from thienyl, furyl, and pyrrolyl;
each R^{A} is independently selected from OH, C₁₋₃ alkoxy, and NH₂;
ring B is pyridinyl, pyrazinyl, and pyrimidinyl; and
each R^{B} is selected from OH, C₁₋₃ alkoxy, SH, NH₂, C₁₋₃ alkylamino, and di(C₁₋₃ alkyl)amino.

18. The compound of claim 14, wherein the compound of Formula (II) is selected from the group consisting of: 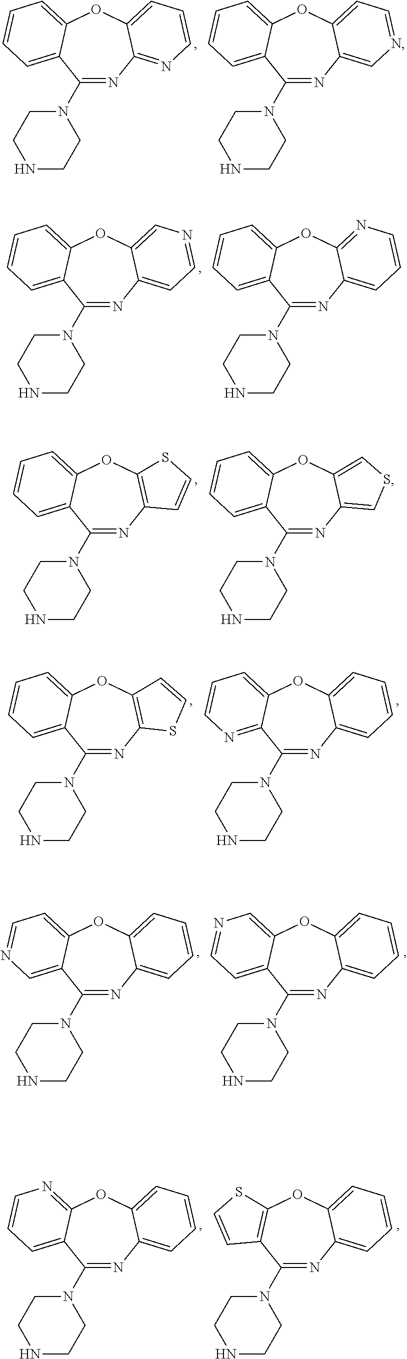 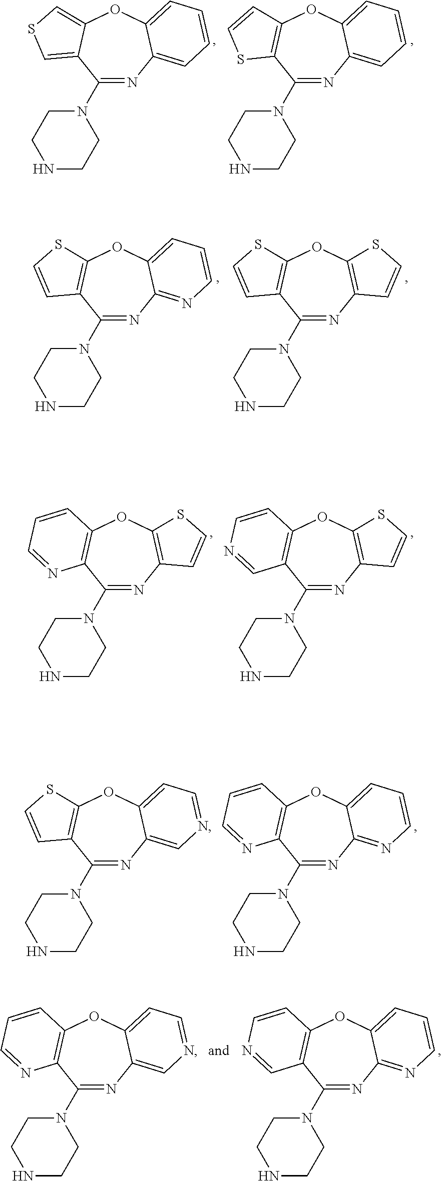
or a pharmaceutically acceptable salt thereof.

19-26. (canceled)
